(19) United States
(12) Patent Application Publication
(10) Pub. No.: US 2012/0204887 A1
(43) Pub. Date: Aug. 16, 2012

(54) Adjustable Snore-Attenuating Pressure (ASAP)

(76) Inventor: Robert A. Connor, Forest Lake, MN (US)

(21) Appl. No.: 13/370,740

(22) Filed: Feb. 10, 2012

Related U.S. Application Data
(60) Provisional application No. 61/463,055, filed on Feb. 11, 2011.

(51) Int. Cl. A61F 5/56

(52) U.S. Cl. 128/848

(57) ABSTRACT

This invention is a device and method to reduce snoring that: is wearable, self-contained, and largely energy self-sufficient; and enables iterative adjustment of airflow characteristics to reduce soft tissue vibration. It is also well-suited to being remotely controlled by the snoring person's bed partner. This invention may be embodied as a device that includes: an airflow-constraining member that directs, reduces, or blocks respiratory airflow during sleep; an airflow channel that allows airflow through or past the airflow-constraining member; and an airflow-adjusting mechanism that enables adjustment of one or more airflow characteristics of airflow through the airflow channel in order to reduce or prevent vibration of soft tissue along the person's airway that causes snoring.

### CROSS-REFERENCE TO RELATED APPLICATIONS

This patent application claims the priority benefit of U.S. Provisional Patent Application No. 61463055 entitled “Snore-Cancelling Device” filed on Feb. 11, 2011 by Robert A. Connor.

### FEDERALLY SPONSORED RESEARCH

Not Applicable

### SEQUENCE LISTING OR PROGRAM

Not Applicable

### BACKGROUND

### Field of Invention

This invention relates to snoring.

### Introduction To Snoring

There are multiple factors that contribute to snoring, but snoring almost always involves vibration of soft tissue along the upper airway. Contributing factors include excess tissue along the airway, extended soft palate or long uvula, poor muscle tone, and airway blockage. Estimates of the percentage of the U.S. population who snore habitually generally range from 20%-30%, with increasing prevalence among men, increasing prevalence with age, and increasing prevalence with increasing obesity.

Some people may view snoring as just an annoyance, but there is increasing evidence that habitual snoring can have serious negative effects on the health and well-being of people who snore, as well as their bed partners. Snoring directly deprives both the snoring person and their bed partner of sleep. On average, habitual snoring deprives both the snorer and their partner of around one hour of sleep per night. Indirectly, there is increasing evidence that snoring can contribute to serious negative health outcomes including atherosclerosis, high blood pressure, heart attack, and stroke.

Beyond these tangible negative health outcomes, snoring can also have very detrimental effects on close interpersonal relationships. This harms well-being and can have negative health effects as well in the long run. Although considerable effort has gone into the development of devices and methods to treat snoring, current devices and methods have significant limitations. Snoring remains untreated and dampens life for tens of millions of people. That is the motivation for this invention that provides a new option for snoring reduction.

### Categorization And Review of the Prior Art

It can often be challenging to classify prior art into discrete categories. That is the case in this field. There are hundreds of examples of potentially-relevant prior art related to this invention, ranging from sound cancelling devices to wearable CPAP systems to dental appliances to air-channeling mouth inserts. However, classification of the prior art into categories, even if imperfect, can be an invaluable tool for reviewing the prior art, identifying its limitations, and setting the stage for discussion of the advantages of the present invention that is disclosed in subsequent sections. Towards this end, I have identified 12 general categories of prior art (including a final miscellaneous category) and identified examples of prior art which appear to be best classified into these categories. This categorization and discussion of the prior art helps to identify key limitations of the prior art which are addressed by the invention disclosed in subsequent sections. The 12 categories of prior art that I will now discuss are as follows: (1) sound cancellation devices and methods for snoring reduction; (2) vibratory airflow devices and methods for clearing the lungs; (3) actively-powered wearable Positive Airway Pressure (PAP) and air filtration devices and methods; (4) passive, airflow resistance and/or acceleration devices and methods; (5) mouth inserts and appliances to reduce or block airflow; (6) mouth inserts and appliances to keep and/or move the tongue forward; (7) tongue implants and surgical procedures; (8) non-tongue soft tissue attachments and implants; (9) sophisticated respiratory sensing and pressure adjustment devices and methods; (10) mouth inserts and appliances to keep and/or move the jaw forward; (11) alarms or other external responses to snoring or other respiratory events; and (12) miscellaneous potentially-relevant prior art.

### 1. Sound Cancellation Devices And Methods For Snoring Reduction

This category of prior art includes sound cancellation devices and methods. These devices and methods are intended to cancel out an undesired first sound wave by producing a second sound wave. The second sound wave is generally the longitudinal-axis mirror image of the first sound wave, so that ideally the two sound waves cancel each other out when they overlap.

Such sound cancellation is easier to implement for continuous or predictable sound patterns than for random or unpredictable sound patterns. Sound cancellation is also easier to implement when the first sound wave is heard along a constant vector or when the source of the second sound wave is very close to the source of the first sound wave. It can be challenging to implement sound cancellation for snoring when snoring is unpredictable. It can also be challenging to implement sound cancellation for snoring because the first sound source is soft tissue inside the human body. The following examples in the prior art disclose application of sound cancellation to snoring or relate to sound cancellation in general that is particularly relevant to snoring applications.

Examples of devices and methods in the prior art that appear to be best classified in this category include the following: U.S. Pat. No. 4,153,815 (Chaplin et al., May 8, 1979, “Active Attenuation of Recurring Sounds”); U.S. Pat. No. 4,473,906 (Warnaka et al., Sep. 25, 1984, “Active Acoustic Attenuator”); U.S. Pat. No. 4,654,871 (Chaplin et al., Mar. 31, 1987, “Method and Apparatus for Reducing Repetitive Noise Entering the Ear”); U.S. Pat. No. 5,133,017 (Cain et al., Jul. 21, 1992, “Noise Suppression System”); U.S. Pat. No. 5,444,786 (Raviv, Aug. 22, 1995, “Snoring Suppression System”); U.S. Pat. No. 5,844,996 (Enzmann et al., Dec. 1, 1998, “Active Electronic Noise Suppression System and Method for Reducing Snoring Noise”); U.S. Pat. No. 6,330,336 (Kasama, Dec. 11, 2001, “Active Silencer”); U.S. Pat. No. 6,431,171 (Burton, Aug. 13, 2002, “Controlling Gas or Drug Delivery to Patient”); and U.S. Pat. No. 7,835,529 (Hernandez et al., Nov. 16, 2010, “Sound Canceling Systems and Methods”); and U.S. patent applications 20030235313 (Kurzweil et al., Dec. 25, 2003, “Sleep-Aide Device”); 20040234080 (Hernandez et al., Nov. 25, 2004, “Sound Canceling Systems and Methods”); 20090147965 (Kuo, Jun. 11, 2009, “Electronic Pillow for Abating Snoring/Environmental Noises, Hands-Free Communications, and Non-Invasive Monitoring and Recording”); 20100283618 (Wolfe et al., Nov. 11, 2010, “Snoring Treatment”); and 20110071444 (Kassatly et al., Mar. 24, 2011, “Discontinuous Positive Airway Pressure Device and Method of Reducing Sleep Disordered Breathing Events”).

### 2. Vibratory Airflow Devices And Methods For Clearing the Lungs

This category of prior art includes vibratory devices and methods that create vibratory airflow to help clear out the lungs. Vibratory airflow is intended to dislodge secretions or other material from the lungs so that this material can be expelled more easily. Devices in this category are often handheld and are used while a person is awake. Although few, if any, of the devices and methods in this category are intended to be used while a person is asleep and/or for treatment of snoring, this category is included in this review because vibratory airflow is potentially relevant.

Examples of devices and methods in the prior art that appear to be best classified in this category include the following U.S. patents: U.S. Pat. No. 4,054,134 (Kritzer, Oct. 18, 1977, “Respirators”); U.S. Pat. No. 4,062,358 (Kritzer, Dec. 13, 1977, “Respirators”); U.S. Pat. No. 6,694,978 (Bennarsten, Feb. 24, 2004, “High-Frequency Oscillation Patient Ventillator System”); U.S. Pat. No. 7,617,821 (Hughes, Nov. 17, 2009, “Acoustic Respiratory Therapy Apparatus”); U.S. Pat. No. 7,779,841 (Dunsmore et al., Aug. 24, 2010, “Respiratory Therapy Device and Method”); U.S. Pat. No. 8,025,054 (Dunsmore et al., Sep. 27, 2011, “Passive Respiratory Therapy Device”); U.S. Pat. No. 8,051,854 (Faram, Nov. 8, 2011, “Continuous High-Frequency Oscillation Breathing Treatment Apparatus”); and U.S. Pat. No. 8,082,920 (Hughes, Dec. 27, 2011, “Acoustic Respiratory Therapy Apparatus”).

### 3. Actively-Powered Wearable Positive Airway Pressure (PAP) And Air Filtration

This category of prior art includes wearable (self-contained) actively-powered devices and methods for Positive Airway Pressure (PAP) and air filtration. This category includes wearable, self-contained devices and methods that provide Continuous Positive Airway Pressure (CPAP). CPAP is used to treat Obstructive Sleep Apnea (OSA) which, in most cases, can have more serious effects on a person's health than snoring. CPAP can also help to reduce snoring. However, many people do not like or cannot tolerate CPAP. Some people do not like having a mask on their face. Other people do not like the continuous pressure. Other people do not like the noise of the air pump. CPAP also has significant energy requirements that eliminate it as an option for people in many areas of the world.

Many people who have Obstructive Sleep Apnea (OSA) would benefit from CPAP for OSA treatment, but do not use CPAP. Since many people who would benefit from CPAP for the potentially more-serious condition of OSA do not use CPAP, the number of people who would benefit from CPAP for snoring but do not use CPAP is probably much greater. For this snoring population, there remains a need for devices and methods to treat snoring that are more palatable than CPAP. In this category, wearable actively-powered air filtration devices have also been included. Although powered self-contained air filtration devices are different in use than powered self-contained CPAP devices, there are a number of common design challenges and elements.

Examples of devices and methods in the prior art that appear to be best classified in this category include the following U.S. patents: U.S. Pat. No. 4,233,972 (Hauff et al., Nov. 18, 1980, “Portable Air Filtering and Breathing Assist Device”); U.S. Pat. No. 4,549,542 (Chien, Oct. 29, 1985, “Multiple-Effect Respirator”); U.S. Pat. No. 4,886,056 (Simpson, Dec. 12, 1989, “Breathing Apparatus”); U.S. Pat. No. 5,035,239 (Edwards, Jul. 30, 1991, “Powered Respirators”); U.S. Pat. No. 5,303,701 (Heins et al., Apr. 19, 1994, “Blower-Supported Gas Mask And Breathing Equipment With An Attachable Control Part”); U.S. Pat. No. 5,372,130 (Stern et al., Dec. 13, 1994, “Face Mask Assembly and Method Having a Fan and Replaceable Filter”); U.S. Pat. No. 6,257,235 (Bowen, Jul. 10, 2001, “Face Mask with Fan Attachment”); U.S. Pat. No. 6,435,184 (Ho, Aug. 20, 2002, “Gas Mask Structure”); U.S. Pat. No. 6,705,314 (O'Dea, Mar. 16, 2004, “Apparatus and Method for Relieving Dyspnoea”); U.S. Pat. No. 6,772,762 (Piesinger, Aug. 10, 2004, “Personal Powered Air Filtration Sterilization and Conditioning System”); U.S. Pat. No. 6,854,464 (Mukaiyama, Feb. 15, 2005, “Respiration Protecting Apparatus”); U.S. Pat. No. 6,895,959 (Lukas, May 24, 2005, “Gas Mask and Breathing Equipment with a Compressor”); U.S. Pat. No. 6,895,962 (Kullik et al., May 24, 2005, “Device for Supporting Respiration”); U.S. Pat. No. 7,19,5014 (Hoffman, Mar. 27, 2007, “Portable Continuous Positive Airway Pressure System”); U.S. Pat. No. 7,195,015 (Kuriyama, Mar. 27, 2007, “Breathing Apparatus”); U.S. Pat. No. 7,464,705 (Tanizawa et al., Dec. 16, 2008, “Powered Respirator”); U.S. Pat. No. 7,516,743 (Hoffman, Apr. 14, 2009, “Continuous Positive Airway Pressure Device and Configuration For Employing Same”); U.S. Pat. No. 7,823,590 (Bibi et al., Nov. 2, 2010, “Devices for Preventing Collapse of the Upper Airway Methods for Use Thereof and Systems and Articles of Manufacture Including Same”); U.S. Pat. No. 7,874,290 (Chalvignac, Jan. 25, 2011, “Breathing Assistance Device”); U.S. Pat. No. 7,913,692 (Kwok, Mar. 29, 2011, “CPAP Mask and System”); U.S. Pat. No. 8,020,556 (Hayek, Sep. 20, 2011, “Respiratory Apparatus”); U.S. Pat. No. 8,020,557 (Bordewick et al., Sep. 20, 2011, “Apparatus and Methods for Administration of Positive Airway Pressure Therapies”); and U.S. Pat. No. 8,069,853 (Tilley, Dec. 6, 2011, “Breath Responsive Powered Air-Purifying Respirator”).

Examples of devices and methods in the prior art that appear to be best classified in this category also include the following U.S. patent applications: 20030066527 (Chen, Apr. 10, 2003, “Face Mask Having Device For Drawing Air Into The Mask”); 20030154983 (Marx, Aug. 21, 2003, “Personal Air Filtering Device”); 20030172930 (Kullik et al., Sep. 18, 2003, “Device For Supporting Respiration”); 20040079373 (Mukaiyama, Apr. 29, 2004, “Respiration Protecting Apparatus”); 20040168689 (Kuriyama, Sep, 2, 2004, “Respirator”); 20040237965 (Bibi et al., Dec. 2, 2004, “Devices for Preventing Collapse of the Upper Airway Methods for Use Thereof and Systems and Articles of Manufacture Including Same”); 20050034724 (O'Dea, Feb. 17, 2005, “Apparatus and Method for Relieving Dyspnoea”); 20060096596 (Occhialini et al., May 11, 2006, “Wearable System for Positive Airway Pressure Therapy”); 20060213516 (Hoffman, Sep. 28, 2006, “Portable Continuous Positive Airway Pressure System”); 20060237013 (Kwok, Oct. 26, 2006, “Ventilator Mask and System”); 20070000493 (Cox, Jan. 4, 2007, “Apparatus for Maintaining Airway Patency”); 20070163600 (Hoffman, Jul. 19, 2007, “User Interface and Head Gear for a Continuous Positive Airway Pressure Device”); 20070246045 (Hoffman, Oct. 25, 2007, “Continuous Positive Airway Pressure Device and Configuration for Employing Same”); 20070251527 (Sleeper, Nov. 1, 2007, “Self-Contained Respiratory Therapy Apparatus for Enhanced Patient Compliance”); 20070277827 (Bordewick et al., Dec. 6, 2007, “Apparatus and Methods for Administration of Positive Airway Pressure Therapies”); 20080178879 (Roberts et al., Jul. 31, 2008, “Impeller for a Wearable Positive Airway Pressure Device”); 20080216831 (McGinnis et al., Sep. 11, 2008, “Standalone CPAP Device and Method of Using”); 20080216835 (McGinnis et al., Sep. 11, 2008, “Standalone CPAP Device and Method of Using”); 20080251079 (Richey, Oct. 16, 2008, “Apparatus and Method for Providing Positive Airway Pressure”); 20100108070 (Kwok, May 6, 2010, “Ventilator Mask and System”); 20100163043 (Hart et al., Jul. 1, 2010, “Self-Contained Oral Ventilation Device”); 20100170513 (Bowditch et al., Jul. 8, 2010, “Self-Contained, Intermittent Positive Airway Pressure Systems and Methods for Treating Sleep Apnea, Snoring, and Other Respiratory Disorders”); and 20120000463 (Bordewick et al., Jan. 5, 2012, “Apparatus and Methods for Administration of Positive Airway Pressure Therapies”).

### 4. Passive Airflow Resistance And/Or Acceleration Devices And Methods

This category of prior art includes wearable, or handheld, passive devices for creating airflow resistance and/or airflow acceleration. Some devices and methods in this category use passive mechanisms to create resistance to air inflow during inhalation. Some devices and methods use passive mechanisms to create resistance to air outflow during exhalation. Some devices and methods in this category use passive mechanisms to accelerate air inflow during inhalation.

Intended uses for devices and methods in this category include treatment of Obstructive Sleep Apnea (OSA) and exercising the lungs (for better respiration after the device is removed). Some of the devices and methods in this category can be as simple as an air tube that restricts airflow during inhalation and/or exhalation. Some of these devices are worn, others are handheld. Handheld devices are not well suited for use while sleeping. Few, if any, of these devices and methods enable a user to program patterns of treatment over the span of multiple respiratory cycles. Few, if any, of these devices and methods employ feedback loops and iterative adjustments to optimally address a respiratory condition or event. Few, if any, offer remote control functionality.

Examples of devices and methods in the prior art that appear to be best classified in this category include the following U.S. patents: U.S. Pat. No. 393,869 (Warren, Dec. 4, 1888, “Inhaler”); U.S. Pat. No. 746,869 (Moulton, Dec. 15, 1903, “Device for Preventing Snoring”); U.S. Pat. No. 957,548 (Doane, May 10, 1910, “Inhaler”); U.S. Pat. No. 1,635,272 (Hartl, Jul. 12, 1927, “Device for Correcting Respiration”); U.S. Pat. No. 3,710,780 (Milch, Jan. 16, 1973, “Respiratory Device with Variable Expiratory Pressure Resistance”); U.S. Pat. No. 3,908,987 (Boehringer, Sep. 30, 1975, “Controlled Positive End Pressure Expiratory Device”); U.S. Pat. No. 4,601,465 (Roy, Jul. 22, 1986, “Device for Stimulating the Human Respiratory System”); U.S. Pat. No. 4,739,987 (Nicholson, Apr. 26, 1988, “Respiratory Exerciser”); U.S. Pat. No. 4,770,413 (Green, Sep. 13, 1988, “Breathing Exercise Device”); U.S. Pat. No. 4,854,574 (Larson et al., Aug. 8, 1989, “Inspirator Muscle Trainer”); U.S. Pat. No. 5,018,517 (Liardet, May 28, 1991, “Expiration-Resisting Apparatus Designed for Improving Pulmonary Ventilation”); U.S. Pat. No.,5,451,190 (Liardet, Sep. 19, 1995, “Apparatus for Respiratory Therapy”); U.S. Pat. No. 5,649,533 (Oren, Jul. 22, 1997, “Therapeutic Respiration Device”); U.S. Pat. No. 6,371,112 (Bibi, Apr. 16, 2002, “Device, System and Method for Preventing Collapse of the Upper Airway”); U.S. Pat. No. 6,595,212 (Arnott, Jul. 22, 2003, “Method and Apparatus for Maintaining Airway Patency”); U.S. Pat. No. 6,629,529 (Arnott, Oct. 7, 2003, “Method for Maintaining Airway Patency”); U.S. Pat. No. 6,763,828 (Arnott, Jul. 20, 2004, “Apparatus for Maintaining Airway Patency”); U.S. Pat. No. 7,506,649 (Doshi et al., Mar. 24, 2009, “Nasal Devices”); U.S. Pat. No. 7,823,590 (Bibi et al., Nov. 2, 2010, “Devices for Preventing Collapse of the Upper Airway Methods for Use Thereof and Systems and Articles of Manufacture Including Same”); U.S. Pat. No. 7,856,979 (Doshi et al., Dec. 28, 2010, “Nasal Respiratory Devices”); U.S. Pat. No. 7,987,852 (Doshi et al., Aug. 2, 2011, “Nasal Devices”); U.S. Pat. No. 7,992,563 (Doshi, Aug. 9, 2011, “Methods and Devices for Improving Breathing in Patients with Pulmonary Disease”); U.S. Pat. No. 7,992,564 (Doshi et al., Aug. 9, 2011, “Respiratory Devices”); U.S. Pat. No. 8,043,236 (Goldshtein et al., Oct. 25, 2011, “Breath Training Device”); and U.S. Pat. No. 8,061,357 (Pierce et al., Nov. 22, 2011, “Adhesive Nasal Respiratory Devices”).

Examples of devices and methods in the prior art that appear to be best classified in this category also include the following: U.S. patent applications 20020104541 (Bibi et al., Aug. 8, 2002, “Devices, Systems and Methods for Preventing Collapse of the Upper Airway and Sensors for Use Therein”); 20030192543 (Arnott, Oct. 16, 2003, “Apparatus for Maintaining Airway Patency”); 20040216741 (Arnott, Nov. 4, 2004, “Apparatus for Maintaining Airway Patency”); 20040237965 (Bibi et al., Dec. 2, 2004, “Devices for Preventing Collapse of the Upper Airway Methods for Use Thereof and Systems and Articles of Manufacture Including Same”); 20070277832 (Doshi et al., Dec. 6, 2007, “Nasal Respiratory Devices”); 20100326447 (Loomas et al., Dec. 30, 2010, “Nasal Respiratory Devices for Positive End-Expiratory Pressure”); 20110005520 (Doshi et al., Jan. 13, 2011, “Quiet Nasal Respiratory Devices”); 20110067709 (Doshi et al., Mar. 24, 2011, “Nasal Respiratory Devices”); 20110218451 (Lai et al., Sep. 8, 2011, “Nasal Devices, Systems and Methods”); and 20110220123 (Robson, Sep. 15, 2011, “Anti-Snoring Device Using Naturally Generated Positive Pressure”).

### 5. Mouth Inserts And Appliances To Reduce Or Block Airflow

This category of prior art includes mouth inserts and dental appliances that selectively reduce or block airflow through the mouth during respiration. These devices can be as simple as a mouth insert that completely blocks airflow through the mouth, but most include some type of opening or channel that restricts airflow through the mouth without blocking it entirely. Few devices and methods in this category give the user an opportunity to selectively adjust the degree of airflow reduction. Few, if any, of these devices and methods respond in an automatic, or iterative, manner to optimally reduce snoring. For snorers for whom uniform reduction or blocking of airflow through the mouth eliminates their snoring and who can tolerate a device that does this constantly, such a device can solve their snoring problem. However, for the many snorers for whom uniform reduction or blocking of airflow through the mouth does not eliminate their snoring or who cannot tolerate a device that does this constantly, there remains an unmet need for devices and methods to reduce snoring.

Examples of devices and methods in the prior art that appear to be best classified in this category include the following: U.S. Pat. No. 5,046,512 (Murchie, Sep. 10, 1991, “Method and Apparatus for Treatment of Snoring”); U.S. Pat. No. 6,089,232 (Portnoy et al., Jul. 18, 2000, “Snore Stopper”); U.S. Pat. No. 6,123,082 (Berthon-Jones, Sep. 26, 2000, “Device for Preventing or Reducing the Passage of Air Through the Mouth”); U.S. Pat. No. 6,263,877 (Gall, Jul. 24, 2001, “Snore Prevention Apparatus”); and U.S. Pat. No. 6,792,942 (Ho et al., Sep. 21, 2004, “Sleep Silencer”); and U.S. patent applications 20060169289 (Zacco, Aug. 3, 2006, “Mouthpiece for Reducing Snoring”); and 20080053459 (Silker, Mar. 6, 2008, “Anti-Snoring Device”).

### 6. Mouth Inserts And Appliances To Keep And/Or Move the Tongue Forward

This category of prior art includes mouth inserts and dental appliances that engage the outside of a person's tongue in order to keep it or move it forward. The intent is to keep the tongue from slipping backwards wherein it might obstruct the upper airway. Such obstruction can be associated with Obstructive Sleep Apnea (OSA), snoring, or both. Some devices seek to engage the tongue with passive suction. Other devices engage the tongue with active suction (negative pressure). Other devices engage the tongue with compressive elements such as clamps, elastic bands, and such. Implementation of these approaches is challenging because the tongue is slippery. The tongue can move and pull out of whatever mechanism is intended to hold it in place. Further, with respect to suction, continuous suction can have detrimental effects on tissue. Finally, for many people, snoring is not corrected by simply holding or moving the tongue forward.

Examples of devices and methods in the prior art that appear to be best classified in this category include the following U.S. patents: U.S. Pat. No. 4,169,473 (Samelson, Oct. 2, 1979, “Anti-Snoring and Anti-Bruxism Device”); U.S. Pat. No. 5,154,184 (Alvarez, Oct. 13, 1992, “Adjustable Anti-Snoring Apparatus”); U.S. Pat. No. 5,465,734 (Alvarez et al., Nov. 14, 1995, “Adjustable Tongue Positioning Device and Method”); U.S. Pat. No. 5,649,540 (Alvarez et al., Jul. 22, 1997, “Tongue Positioning Device for Medical Procedures”); U.S. Pat. No. 5,666,973 (Walter, Sep. 16, 1997, “Device to Reduce or Prevent Night Clenching and Grinding of Teeth and Snoring”); U.S. Pat. No. 5,957,133 (Hart, Sep. 28, 1999, “Oral Appliance with Negative Air Supply for Reducing Sleep Apnea and Snoring”); U.S. Pat. No. 5,988,170 (Thomas, Nov. 23, 1999, “Snoring Prevention Apparatus”); U.S. Pat. No. 6,055,986 (Meade, May 2, 2000, “Apparatus and Method for the Reduction of Snoring”); U.S. Pat. No. 6,494,209 (Kulick, Dec. 17, 2002, “Method and Apparatus for Treatment of Snoring, Hypopnea and Apnea”); U.S. Pat. No. 6,877,513 (Scarberry et al., Apr. 12, 2005, “Intraoral Apparatus for Enhancing Airway Patency”); U.S. Pat. No. 7,137,393 (Pivovarov, Nov. 21, 2006, “Breathing Normalizer Apparatus”); U.S. Pat. No. 7,861,724 (Keropian, Jan. 4, 2011, “Sleep Appliance”); U.S. Pat. No. 7,954,494 (Connor, Jun. 7, 2011, “Device with Actively-Moving Members that Hold or Move the Tongue”); and U.S. Pat. No. 8,091,554 (Jiang, Jan. 10, 2012, “Methods and Devices for Relieving Upper Airway Obstructions”).

Examples of devices and methods in the prior art that appear to be best classified in this category also include the following U.S. patent applications: 20110073119 (Chen et al., Mar. 31, 2011, “Negative Pressure Oral Apparatus”); 20110155143 (Shantha, Jun. 30, 2011, “Snoring and Obstructive Sleep Apnea Prevention and Treatment Device”); 20110180075 (Chen et al., Jul. 28, 2011, “Adjustable Oral Interface and Method to Maintain Upper Airway Patency”); 20110192404 (Chen, Aug. 11, 2011, “Automated Negative Pressure Oral Apparatus”); 20110265801 (Cullen, Nov. 3, 2011, “Device for the Alleviation of Snoring and Sleep Apnoea”); and 20120017917 (Podmore et al., Jan. 26, 2012, “Airway Device with Tongue-Engaging Member”).

### 7. Tongue Implants And Surgical Procedures

This category of prior art includes tongue implants and surgical procedures to keep the tongue in a forward position within the mouth. Implants include tissue anchors, sutures, and stents. In this respect, the intent of devices and methods in this category is similar to that of devices and methods in the previous category. These devices and methods are intended to keep the tongue from slipping backwards wherein it might obstruct the upper airway. Compared to the previous category, the use of internal implants or surgical devices can overcome the problems with engaging the slippery outside of the tongue. It is easier to grip tongue tissue from inside.

However, this category also has some disadvantages compared to the previous category. First, most tongue implants are permanent. A person is not able to insert them during sleep and then remove them during the day. Accordingly, the tongue implant may adversely affect speech or eating during the day. Second, implants and surgical procedures that involve the interior of the tongue are more invasive than mouth inserts that engage only the outside of the tongue. Invasive surgical procedures tend to be more expensive and have more risks than non-invasive approaches. Finally, as with the previous category, there is no guarantee that snoring will be corrected by holding the tongue forward.

Examples of devices and methods in the prior art that appear to be best classified in this category include the following: U.S. Pat. No. 7,658,192 (Harrington, Feb. 9, 2010, “Method and Device for Treatment of Obstructive Sleep Apnea”); U.S. Pat. No. 7,909,038 (Hegde et al., Mar. 22, 2011, “Tongue Stabilization Device and Methods of Using the Same”); U.S. Pat. No. 7,921,850 (Nelson et al., Apr. 12, 2011, “Systems and Methods for Moving and/or Restraining Tissue in the Upper Respiratory System”); and U.S. Pat. No. 8,074,655 (Sanders, Dec. 13, 2011, “Methods and Devices for Treating Sleep Apnea and Snoring”); and U.S. patent applications 20080188947 (Sanders, Aug. 7, 2008, “Methods and Devices for Treating Sleep Apnea and Snoring”); and 20100139668 (Harrington, Jun. 10, 2010, “Method and Device for Treatment of Obstructive Sleep Apnea”).

### 8. Non-Tongue Soft Tissue Attachments And Implants

This category of prior art includes devices that are attached to, or implanted within, soft tissue along the upper airway other than the tongue. Most of the devices and methods in this category stiffen the soft tissue so that it does not vibrate. These devices and methods are promising but have limitations. It can be difficult to attach materials to the outside of the soft tissue because of the gag reflex and because the soft tissue is slippery. Implanting devices within soft tissue is relatively permanent and invasive. Also, devices and methods in this category in the prior art do not appear to be adjustable once attached or inserted.

Examples of devices and methods in the prior art that appear to be best classified in this category include the following: U.S. Pat. No. 6,467,485 (Schmidt, Oct. 22, 2002, “Anti-Snoring Device and Method”); U.S. Pat. No. 6,748,951 (Schmidt, Jun. 15, 2004, “Anti-Snoring Devices and Methods”); U.S. Pat. No. 7,992,566 (Pflueger et al. Aug. 9, 2011, “Apparatus and Methods for Treating Sleep Apnea”); and U.S. Pat. No. 8,037,885 (Metzger et al., Oct. 18, 2011, “Treatment for Sleep Apnea or Snoring”); and U.S. patent applications 20030062050 (Schmidt, Apr. 3, 2003, “Anti-Snoring Devices and Methods”); 20050279365 (Armijo et al., Dec. 22, 2005, “Anti-Snoring Apparatus and Method”); and 20110290258 (Pflueger et al., Dec. 1, 2011, “Apparatus and Methods for Treating Sleep Apnea”).

### 9. Sophisticated Respiratory Sensing And Pressure Adjustment Devices And Methods

This category of prior art includes sophisticated respiratory sensing and positive pressure adjustment devices and methods. Most of these devices and methods include sensors and feedback loops in order to fine tune therapy. These devices and methods tend to be complex. Most of this art relates to adjustment of CPAP, or other Positive Airway Pressure (PAP) therapies, in response to changes in human respiratory cycles and events.

In some respects, the boundaries of this category are less distinct than those of other categories in this review because the adjective “sophisticated” is subjective. Almost all CPAP systems involve some type of adaptive pressure-changing mechanism. Nonetheless, it is important to include this as a discrete category in order to highlight prior art that adjusts airflow therapy, in an intelligent manner, in accordance with sensor feedback concerning human respiratory cycles and events. It is particularly relevant because airflow therapy provided by such devices and methods can be automatic, iterative, and adaptive.

Examples of devices and methods in the prior art that appear to be best classified in this category include the following U.S. patents: U.S. Pat. No. 5,134,995 (Gruenke et al., Aug. 4, 1992, “Inspiratory Airway Pressure System with Admittance Determining Apparatus and Method”); U.S. Pat. No. 5,148,802 (Sanders et al., Sep. 22, 1992, “Method and Apparatus for Maintaining Airway Patency to Treat Sleep Apnea and Other Disorders”); U.S. Pat. No. 5,199,424 (Sullivan et al., Apr. 6, 1993, “Device for Monitoring Breathing During Sleep and Control of CPAP Treatment that is Patient Controlled”); U.S. Pat. No. 5,203,343 (Axe et al., Apr. 20, 1993, “Method and Apparatus for Controlling Sleep Disorder Breathing”); U.S. Pat. No. 5,245,995 (Sullivan et al., Sep. 21, 1993, “Device and Method for Monitoring Breathing During Sleep, Control of CPAP Treatment, and Preventing of Apnea”); U.S. Pat. No. 5,259,373 (Gruenke et al. Nov. 9, 1993, “Inspiratory Airway Pressure System Controlled by the Detection and Analysis of Patient Airway Sounds”); U.S. Pat. No. 5,301,689 (Wennerholm, Apr. 12, 1994, “Device for Temporary Artificial Respiration Assistance for Persons Having Snore Problems”); U.S. Pat. No. 5,433,193 (Sanders et al., Jul. 18, 1995, “Breathing Gas Delivery Method and Apparatus”); U.S. Pat. No. 5,490,502 (Rapoport et al., Feb. 13, 1996, “Method and Apparatus for Optimizing the Continuous Positive Airway Pressure for Treating Obstructive Sleep Apnea”); U.S. Pat. No. 5,535,738 (Estes et al., Jul. 16, 1996, “Method and Apparatus for Providing Proportional Positive Airway Pressure to Treat Sleep Disordered Breathing”); U.S. Pat. No. 5,546,933 (Rapoport et al., Aug. 20, 1996, “Method for Optimizing the Continuous Positive Airway Pressure for Treating Obstructive Sleep Apnea”); U.S. Pat. No. 5,803,066 (Rapoport et al., Sep. 8, 1998, “Method and Apparatus for Optimizing the Continuous Positive Airway Pressure for Treating Obstructive Sleep Apnea”); and U.S. Pat. No. 5,845,636 (Gruenke et al., Dec. 8, 1998, “Method and Apparatus for Maintaining Patient Airway Patency”).

Examples of devices and methods in the prior art that appear to be best classified in this category also include the following U.S. patents: U.S. Pat. No. 5,953,713 (Behbehani et al., Sep. 14, 1999, “Method and Apparatus for Treatment of Sleep Disorder Breathing Employing Artificial Neural Network”); U.S. Pat. No. 6,085,747 (Axe et al., Jul. 11, 2000, “Method and Apparatus for Controlling Sleep Disorder Breathing”); U.S. Pat. No. 6,305,374 (Zdrojkowski et al., Oct. 23, 2001, “Breathing Gas Delivery Method and Apparatus”); U.S. Pat. No. 6,349,724 (Burton et al., Feb. 26, 2002, “Dual-Pressure Blower for Positive Air Pressure Device”); U.S. Pat. No. 6,427,689 (Estes et al., Aug. 6, 2002, “Sleep Apnea Treatment Apparatus”); U.S. Pat. No. 6,467,477 (Frank et al., Oct. 22, 2002, “Breath-Based Control of a Therapeutic Treatment”); U.S. Pat. No. 7,080,645 (Genger et al., Jul. 25, 2006, “Anti-Snoring Device, Method for Reducing Snoring, and a Nasal Air Cannula”); U.S. Pat. No. 7,222,623 (DeVries et al., May 29, 2007, “Portable Drag Compressor Powered Mechanical Ventilator”); U.S. Pat. No. 7,246,619 (Truschel et al., Jul. 24, 2007, “Snore Detecting Method and Apparatus”); U.S. Pat. No. 7,575,005 (Mumford et al., Aug. 18, 2009, “Mask Assembly with Integrated Sensors”); U.S. Pat. No. 7,607,432 (Sullivan, Oct. 27, 2009, “Apparatus and Method for The Treatment of an Upper Airway Flow Limitation”); U.S. Pat. No. 7,793,660 (Kimmel et al., Sep. 14, 2010, “Method of Treating Obstructive Sleep Apnea”); U.S. Pat. No. 7,849,854 (DeVries et al., Dec. 14, 2010, “Portable Drag Compressor Powered Mechanical Ventilator”); U.S. Pat. No. 7,882,834 (Gradon et al., Feb. 8, 2011, “Autotitrating Method and Apparatus”); U.S. Pat. No. 7,938,114 (Matthews et al., May 10, 2011, “Auto-Titration Bi-Level Pressure Support System and Method of Using Same”); U.S. Pat. No. 8,011,365 (Douglas et al., Sep. 6, 2011, “Mechanical Ventilation in the Presence of Sleep Disordered Breathing”); and U.S. Pat. No. 8,020,555 (Rapoport, Sep. 20, 2011, “System and Method for Improved Treatment of Sleeping Disorders Using Therapeutic Positive Airway Pressure”).

Examples of devices and methods in the prior art that appear to be best classified in this category further include the following U.S. patent applications: 20050016536 (Rapoport et al., Jan. 27, 2005, “Method and Apparatus for Optimizing the Continuous Positive Airway Pressure for Treating Obstructive Sleep Apnea”); 20050224078 (Zdrojkowski et al., Oct. 13, 2005, “Breathing Gas Delivery Method and Apparatus”); 20060000475 (Matthews et al., Jan. 5, 2006, “Auto-Titration Bi-Level Pressure Support System and Method of Using Same”); 20070016093 (Rapoport et al., Jan. 18, 2007, “Method and Apparatus for Optimizing the Continuous Positive Airway Pressure for Treating Obstructive Sleep Apnea”); 20110284003 (Douglas et al., Nov. 24, 2011, “Mechanical Ventilation in the Presence of Sleep Disordered Breathing”); and 20120010519 (Rapoport et al., Jan. 12, 2012, “System and Method for Diagnosis and Treatment of Obstructive Sleep Apnea”).

### 10. Mouth Inserts And Appliances To Keep And/Or Move the Jaw Forward

This category of prior art includes mouth inserts and dental appliances that keep or move the jaw forward. The intent is to shift the tongue, and other soft tissue, forward so that it is less likely to obstruct the upper airway. The intent is to address Obstructive Sleep Apnea (OSA), snoring, or both. On the upside, it is relatively easy to move the jaw forward. It is much easier, for example, to engage teeth than it is to engage the outer surface of the tongue. On the downside, however, for many people simply moving their jaw forward does not reduce their snoring.

Examples of devices and methods in the prior art that appear to be best classified in this category include the following: U.S. Pat. No. 5,678,567 (Thornton et al., Oct. 21, 1997, “Method and Apparatus for Adjusting a Dental Device”); U.S. Pat. No. 5,752,510 (Goldstein, May 19, 1998, “Nasal and Oral Air Passageway Delivery Management Apparatus”); U.S. Pat. No. 5826,579 (Remmers et al., Oct. 27, 1998, “Remote-Controlled Mandibular Positioning Device and Method of Using the Device”); U.S. Pat. No. 5,921,942 (Remmers et al., Jul. 13, 1999, “Adaptively Controlled Mandibular Positioning Device and Method of Using the Device”); U.S. Pat. No. 5,954,048 (Thornton, Sep. 21, 1999, “Device and Method for Improving Breathing”); U.S. Pat. No. 5,983,892 (Thornton, Nov. 16, 1999, “Device for Improving Breathing”); U.S. Pat. No. 6,155,262 (Thornton et al., Dec. 5, 2000, “Method and Apparatus for Adjusting a Dental Device”); U.S. Pat. No. 6,273,859 (Remmers et al., Aug. 14, 2001, “Adaptively Controlled Mandibular Positioning Device and Method of Using the Device”); U.S. Pat. No. 6,305,376 (Thornton, Oct. 23, 2001, “Device and Method for Improving Breathing”); U.S. Pat. No. 6,374,824 (Thornton, Apr. 23, 2002, “Device for Improving Breathing”); U.S. Pat. No. 6,845,774 (Gaskell, Jan. 25, 2005, “Dental Device”); and U.S. Pat. No. 8,028,703 (Moses, Oct. 4, 2011, “Sleep Mouthpiece”); and U.S. patent applications 20050081859 (Scarberry et al., Apr. 21, 2005, “Intraoral Apparatus for Enhancing Airway Patency”); 20050236003 (Meader, Oct. 27, 2005, “Apnea Nipple and Oral Airway and Mandibular Advancement Device”); 20090078273 (Bhat et al., Mar. 26, 2009, “Smart Mandibular Repositioning System”); and 20110259345 (Cullen, Oct. 27, 2011, “Device for the Alleviation of Snoring and Sleep Apnoea”).

### 11. Alarms Or Other External Responses To Snoring Or Other Respiratory Events

This category of prior art includes alarms and other external (outside the body) responses to snoring, airway obstruction, or other respiratory events. In addition to alarms, examples of external responses in this category include: automated movement of a mattress or pillow to provoke a change in position by the sleeping person; suction (negative pressure) applied to a portion, such as the neck, of the sleeping person's body; and electrical stimulation applied to part of the sleeping person's body. Such external approaches have potential advantages. For example, they are either minimally invasive or non-invasive. However, there are many people for whom they do not work well to reduce snoring. Vibration of soft tissue that is often the source of snoring is deep within the body and may not be well addressed by external responses. Also, there are people whose sleep is disturbed by being shocked or prodded. Accordingly, there remains a need for other devices and methods for snoring reduction.

Examples of devices and methods in the prior art that appear to be best classified in this category include the following: U.S. Pat. No. 6,371,120 (Chiu et al., Apr. 16, 2002, “Snore Elimination Device”); U.S. Pat. No. 6,386,201 (Fard, May 14, 2002, “Apparatus for Preventing Snoring”); U.S. Pat. No. 6,894,427 (Alfini, May 17, 2005, “Nasal Vibration Transducer”); U.S. Pat. No. 7,513,003 (Mossbeck, Apr. 7, 2009, “Anti-Snore Bed Having Inflatable Members”); U.S. Pat. No. 7,522,062 (Mossbeck, Apr. 21, 2009, “Anti-Snore Bedding Having Adjustable Portions”); U.S. Pat. No. 7,716,988 (Ariav et al., May 18, 2010, “Apparatus for Use in Controlling Snoring and Sensor Unit Particularly Useful Therein”); U.S. Pat. No. 7,789,837 (Lehrman et al., Sep. 7, 2010, “System and Method for Treating Obstructive Sleep Apnea”); U.S. Pat. No. 7,793,661 (Macken, Sep. 14, 2010, “Method and Apparatus for Treatment of Snoring and Sleep Apnea”); and U.S. Pat. No. 7,866,212 (Ariav et al., Jan. 11, 2011, “High-Sensitivity Sensors for Sensing Various Physiological Phenomena, Particularly Useful in Anti-Snoring Apparatus and Methods”); and U.S. patent applications 20070277835 (Ariav et al., Dec. 6, 2007, “Apparatus for use in Controlling Snoring and Sensor Unit Particularly Useful Therein”); and 20080195173 (Villa, Aug. 14, 2008, “Method and Apparatus for Treating Sleep Apnea and Snoring”).

### 12. Miscellaneous Potentially-Relevant Prior Art

There is some prior art that is potentially-relevant to the present invention that is difficult to classify in a particular category. Some of this art relates to portable, but not wearable, CPAP systems. Some of this art relates to application of sound cancellation to air pump noise instead of snoring per se. In the interest of completeness, this potentially-relevant prior art is included here in a “miscellaneous” category. Other prior art, particularly some of the applications that have not yet been approved, is very general and hard to categorize. Although we have not been able to categorize this art into the above categories, we do provide patent titles for each example so that the reader has some general information concerning the focus of each patent to guide their in-depth investigation of any of them as so desired.

Examples of devices and methods in the prior art that appear to be best classified in this category include the following U.S. patents: U.S. Pat. No. 4,076,021 (Thompson, Feb. 28, 1978, “Positive Pressure Respiratory Apparatus”); U.S. Pat. No. 4,298,023 (McGinnis, Nov. 3, 1981, “Spring Loaded Exhalation Valve”); U.S. Pat. No. 4,944,310 (Sullivan, Jul. 31, 1990, “Device for Treating Snoring Sickness”); U.S. Pat. No. 5,657,752 (Landis et al., Aug. 19, 1997, “Nasal Positive Airway Pressure Mask and Method”); U.S. Pat. No. 5,711,296 (Kolobow, Jan. 27, 1998, “Continuous Positive Airway Pressure System”); U.S. Pat. No. 5,884,625 (Hart, Mar. 23, 1999, “Oral Appliance for Delivering Gas to the Retroglossal Area”); U.S. Pat. No. 5,950,624 (Hart, Sep. 14, 1999, “Oral Appliance Having Hollow Body”); U.S. Pat. No. 6,000,396 (Melker et al., Dec. 14, 1999, “Hybrid Microprocessor Controlled Ventilator Unit”); U.S. Pat. No. 6,474,339 (Grosbois et al., Nov. 5, 2002, “Device for Preventing Snoring and Apnoea During Sleep”); U.S. Pat. No. 6,562,057 (Santin, May 13, 2003, “Nasal Breathing Assist Devices”); U.S. Pat. No. 7,448,382 (Alexander et al., Nov. 11, 2008, “Pressure Support System with Active Noise Cancellation”); U.S. Pat. No. 7,890,193 (Tingey, Feb. 15, 2011, “Oral Device”); U.S. Pat. No. 7,909,035 (Thornton, Mar. 22, 2011, “Multi-Chamber Mask and Method of Forming the Same”); and U.S. Pat. No. 8,011,362 (Adams, Sep. 6, 2011, “Compact Continuous Positive Airway Pressure Apparatus and Method”).

Examples of devices and methods in the prior art that appear to be best classified in this category also include the following U.S. patent applications: 20040226562 (Bordewick, Nov. 18, 2004, “Blower Assembly for CPAP”); 20070084470 (Sarazen, Apr. 19, 2007, “Anti-Snoring Apparatus”); 20070215156 (Kwok, Sep. 20, 2007, “Snoring Treatment Apparatus and Methods of Managing Snorers”); 20080029098 (Ottestad, Feb. 7, 2008, “Portable Breathing Apparatus”); 20080110459 (Farbarik, May 15, 2008, “Systems and Methods for Providing Active Noise Control in a Breathing Assistance System”); 20090082839 (Lindquist et al., Mar. 26, 2009, “Electronic Anti-Snoring And Sleep Apnea Device For Sleep-Breathing Disorders, Electronic Anti-Bruxing Device, and Electronic Device For TMD Therapy”); and 20110295083 (Doelling et al., Dec. 1, 2011, “Devices, Systems, and Methods for Monitoring, Analyzing, and/or Adjusting Sleep Conditions”).

### SUMMARY OF THE PRIOR ART

Since habitual snoring seriously impacts many millions of people, there are many examples of prior art that are intended to reduce snoring. Although there are multiple categories, and hundreds of examples, of devices and methods in the prior art that are intended to reduce snoring, there remains an unmet need for snoring reduction. Millions of people still snore. Both they and their bed partners suffer the consequences. There are limitations to all of the snoring devices and methods in the prior art. Many of the approaches in the prior art do not correct snoring at its source—the vibration of soft tissue along the upper airway. One approach that can correct snoring at its source is Continuous Positive Airway Pressure (CPAP). However, many people do not use CPAP to treat the more-serious health condition of Obstructive Sleep Apnea (OSA) because they cannot tolerate CPAP. These people are even less likely to use CPAP to treat the less-serious condition of snoring. They need a more-palatable device and method to reduce the soft tissue vibration that causes snoring.

### SUMMARY OF THIS INVENTION

The prior art does not disclose a wearable self-contained device to reduce snoring: that enables adjustment of airflow characteristics over multiple respiratory cycles to optimally reduce soft tissue vibration; that is largely energy self-sufficient; and that can be remotely controlled by the snoring person's bed partner. Such a device can improve the lives of tens of millions of people. The invention disclosed herein comprises such a device and method and can meet this unmet need for snoring mitigation. This invention is particularly well-suited for use with a wireless remote control which may be operated by the snorer's bed partner as part of a system to find the optimal airflow characteristics that reduce snoring. This invention provides a new option for snoring reduction that can help millions of people to stop snoring, benefiting not only them but their bed partners as well.

This invention may be embodied as a device for reducing snoring that includes: (1) an airflow-constraining member that directs, reduces, or blocks respiratory airflow during sleep; (2) an airflow channel that allows airflow through or past the airflow-constraining member; and (3) an airflow-adjusting mechanism that enables adjustment of one or more airflow characteristics of airflow through the airflow channel in order to reduce or prevent vibration of soft tissue along the person's airway that causes snoring.

Airflow characteristics may be selected from the group consisting of: airflow speed, airflow rate, airflow pressure, airflow volume, airflow duration, airflow timing, airflow direction, the creation of one or more pulses of air, the number of air pulses, pattern of air pulses, the frequency of air pulses, the magnitude of air pulses, and the duration of air pulses. Activation of adjustment of these airflow characteristics may be manual or automatic. The airflow-adjusting mechanism may enable adjustment of airflow characteristics in an iterative manner in order to select airflow characteristics that are successful in reducing or preventing snoring. Further, the airflow-adjusting mechanism may transduce or harvest some of the kinetic energy of airflow from human respiration in order to adjust airflow characteristics in order to reduce the vibration of soft tissue. This helps to make the device and method energy self-sufficient. The airflow-adjusting mechanism may also be remotely controlled, within safety parameters, by the snoring person's bed partner.

### INTRODUCTION TO THE FIGURES

FIGS. 1-29 show examples of how this invention may be embodied, but they do not limit the full generalizability of the claims.

FIG. 1 shows a view of a person's head during inhalation, without any snoring, to provide context for the invention shown in later figures.

FIG. 2 shows this same view, with snoring.

FIG. 3 shows an example of this invention comprising a mouth mask and inflatable airflow-adjusting members, before snoring has been stopped.

FIG. 4 shows an example of this invention comprising a mouth mask and inflatable airflow-adjusting members, after snoring has been stopped.

FIG. 5 shows an example of this invention comprising a mouth mask and automatic inflatable airflow-adjusting members, before snoring has been stopped.

FIG. 6 shows an example of this invention comprising a mouth mask and automatic inflatable airflow-adjusting members, after snoring has been stopped.

FIG. 7 shows an example of this invention comprising a mouth mask and a rotating airflow-adjusting member, before snoring has been stopped.

FIG. 8 shows an example of this invention comprising a mouth mask and a rotating airflow-adjusting member, after snoring has been stopped.

FIG. 9 shows an example of this invention comprising a mouth mask and an automatic rotating airflow-adjusting member, before snoring has been stopped.

FIG. 10 shows an example of this invention comprising a mouth mask and an automatic rotating airflow-adjusting member, after snoring has been stopped.

FIG. 11 shows an example of this invention comprising a mouth mask and an automatic rotating airflow-adjusting member, before snoring has been stopped by the creation of vibratory airflow.

FIG. 12 shows an example of this invention comprising a mouth mask and an automatic rotating airflow-adjusting member, after snoring has been stopped by the creation of vibratory airflow.

FIG. 13 shows an example of this invention comprising a face mask and an automatic rotating airflow-adjusting member, before snoring has been stopped.

FIG. 14 shows an example of this invention comprising a face mask and an automatic rotating airflow-adjusting member, after snoring has been stopped.

FIG. 15 shows an example of this invention comprising a face mask and an automatic rotating airflow-adjusting member, wherein this invention is harvesting energy from airflow during exhalation.

FIG. 16 shows an example of this invention comprising a mouth insert and automatic inflatable airflow-adjusting members, before snoring has been stopped.

FIG. 17 shows an example of this invention comprising a mouth insert and automatic inflatable airflow-adjusting members, after snoring has been stopped.

FIG. 18 shows an example of this invention comprising a mouth mask, rotating airflow-adjusting member, and spring, wherein this invention is harvesting energy from airflow during exhalation.

FIG. 19 shows an example of this invention comprising a mouth mask, rotating airflow-adjusting member, and spring, during inhalation before snoring has been stopped.

FIG. 20 shows an example of this invention comprising a mouth mask, rotating airflow-adjusting member, and spring, during inhalation after snoring has been stopped.

FIG. 21 shows an example of this invention comprising a mouth mask, rotating airflow-adjusting member, and spring, wherein this invention is harvesting energy from airflow during exhalation.

FIG. 22 shows an example of this invention comprising a mouth mask, rotating airflow-adjusting member, and spring, before snoring has been stopped by the creation of vibratory airflow.

FIG. 23 shows an example of this invention comprising a mouth mask, rotating airflow-adjusting member, and spring, after snoring has been stopped by the creation of vibratory airflow.

FIG. 24 shows an example of this invention comprising a mouth mask and multiple airflow channels, before snoring has been stopped.

FIG. 25 shows an example of this invention comprising a mouth mask and multiple airflow channels, after snoring has been stopped.

FIG. 26 shows an example of this invention comprising a mouth mask and an oscillating airflow-adjusting member, before snoring has been stopped.

FIG. 27 shows an example of this invention comprising a mouth mask and an oscillating airflow-adjusting member, after snoring has been stopped.

FIG. 28 shows an example of this invention with a wireless remote control, before snoring has been stopped.

FIG. 29 shows an example of this invention with a wireless remote control, after snoring has been stopped.

### DETAILED DESCRIPTION OF THE FIGURES

The following figures, FIGS. 1-29, show various examples of how this invention may be embodied in a device and method to reduce or prevent snoring. However, these examples are not exhaustive and do not limit the full generalizability of the claims.

FIG. 1 shows a lateral cross-sectional view of a person's head. This view includes a cross-sectional view of the upper respiratory airway during normal inhalation, without any snoring. This figure does not show an example of the invention, but rather provides anatomical and physiological context for the examples of the invention that will be shown in later figures. FIG. 1 shows the main portion of the person's head 101, soft tissue 102 along the person's upper airway, and the person's tongue 103, chin 104, nose 105, and jaw 106.

FIG. 1 also shows nasal inflow 107 which is represented by a dotted line. Nasal inflow is airflow moving inward through the person's nose during inhalation. Nasal inflow 107 is represented in these figures by a dotted line with an end arrow that points inward toward the person's lungs. In this example, nasal inflow draws ambient air inwards through the person's nose. In this example, the dotted line that represents nasal inflow 107 is curvaceous, but relatively smooth. This represents that nasal inflow during normal inhalation is relatively smooth, non-vibratory, and unobstructed.

FIG. 1 also shows oral inflow 108 which is represented by a dotted line. Oral inflow is airflow moving inward through the person's mouth during inhalation. Oral inflow 108 is represented in these figures by a dotted line with an end arrow that points inward toward the person's lungs. In this example, oral inflow draws ambient air inwards through the person's mouth. In this example, the dotted line that represents oral inflow 108 is curvaceous, but relatively smooth. This represents that oral inflow during normal inhalation is relatively smooth, non-vibratory, and unobstructed.

It is important to note that in FIG. 1, during normal inhalation without snoring, the soft tissue 102 along the upper airway is not vibrating. Also, soft tissue 102 is not obstructing the inflow of air through the airway. In this figure, the person is neither snoring nor experiencing airway blockage due to Obstructive Sleep Apnea (OSA). In FIG. 1, the person's head is shown in an upright position with respect to the portrait axis of the paper on which the figure is displayed. However, it is to be understood that this figure can represent the head of a person who is reclining and sleeping.

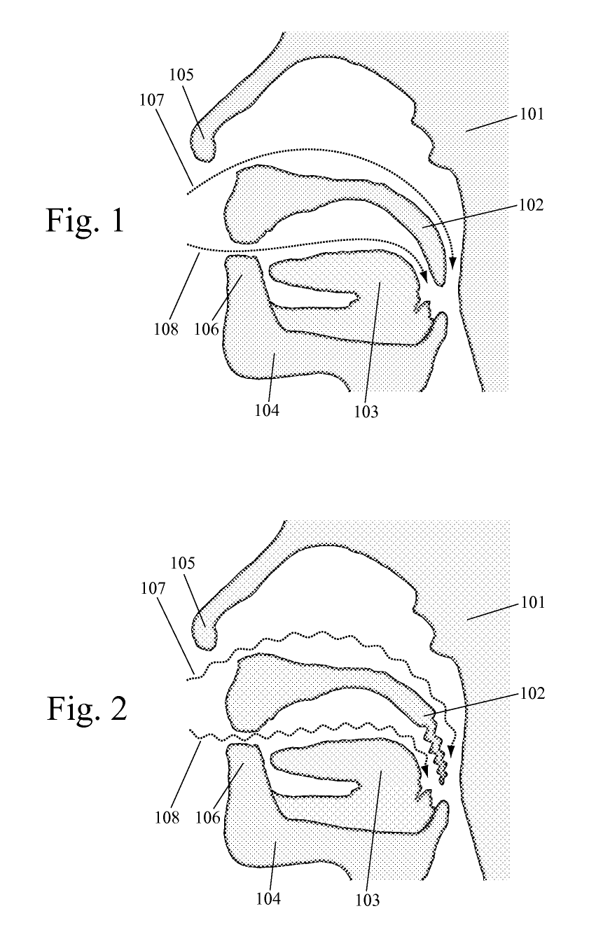

FIG. 2 shows the same lateral cross-sectional view of a person's head that was shown in FIG. 1, but now soft tissue 102 is vibrating, nasal inflow 107 is vibrating, and oral inflow 108 is vibrating. This indicates that the person is snoring. The combined aerodynamics of nasal inflow 107 and oral inflow 108 traveling past both sides of soft tissue 102 have caused soft tissue 102 to flutter at a resonant frequency. This fluttering creates air vibrations that travel upstream through the nasal and oral cavities and exit the person's body as snoring sounds.

In some examples, the occurrence of snoring in FIG. 2 but not in FIG. 1 can be due to differences in anatomy between two people. For example, these different figures may represent two different people who have differences in upper airway anatomy. In other situations, the occurrence of snoring may be triggered by changes in muscle tone, sleep state, or posture over time, within the same person.

The occurrence of snoring in FIG. 2 but not in FIG. 1 can also be due to changes in the combined aerodynamics of nasal inflow 107 and oral inflow 108 as they travel past soft tissue 102. This is the primary focus of this invention for reducing snoring. Certain airflow characteristics, or combinations of these airflow characteristics, of nasal inflow 107 and oral inflow 108 can combine to cause soft tissue 102 to vibrate or flutter at a resonant frequency. This motion can be compared to the vibration of a reed in a woodwind musical instrument or to the oscillation of a span of a suspension bridge in heavy crosswind. These airflow characteristics may include the rate, pressure, volume, and duration of nasal inflow 107 or oral inflow 108, or interaction between the airflow characteristics of nasal inflow 107 and oral inflow 108. This invention reduces snoring by adjusting these airflow characteristics during inhalation to prevent vibration of soft tissue along the airway, such as that shown in FIG. 2.

The vibration, resonance, or fluttering of soft tissue along the airway, such as that shown in FIG. 2, may be caused by differences in the relative speeds, pressures, and/or volumes of nasal and oral airflows, 107 and 108, as they flow past different sides of soft tissue 102 along the airway during inhalation. Trigger-specific air speeds, pressures, and/or volumes of airflows going past the two sides of this soft tissue, or the relative differences between these airflows, can cause fluttering and/or harmonic resonance of the soft tissue. This, in turn, can cause snoring. Adjusting air speeds, pressures, and/or volumes away from these trigger-specific air speeds, pressures, and/or volumes, or the relative differences between airflows, can disrupt the fluttering and/or harmonic resonance of soft tissue along the airway. This can disrupt or cancel snoring at its source.

In various examples, one can change the relative speeds, pressures, and/or volumes of airflows through the mouth vs. nose, respectively, by changing airflow through the mouth only, through the nose only, or through both the nose and the mouth. In an example, one may selectively adjust airflow through the mouth in a different manner than the manner in which one adjusts airflow through the nose. There are potential drawbacks to limiting airflow through the nose. One should not rely on unassisted mouth breathing alone to provide fresh air to a sleeping person because the mouth can close.

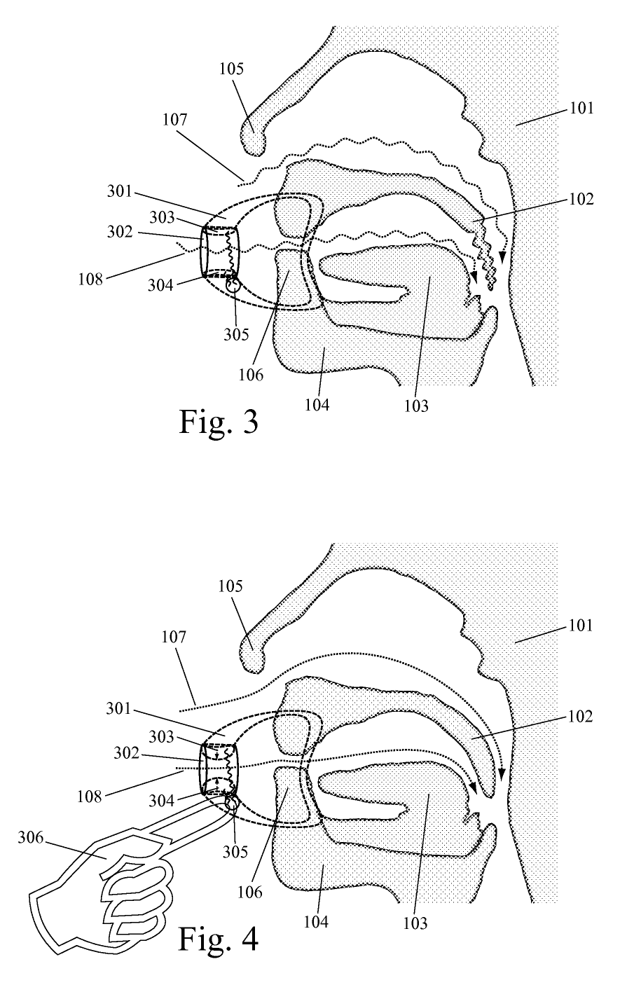

FIG. 3 shows the same lateral cross-sectional view of a person's head that was shown in FIG. 2, with the addition of one example of how this invention may be embodied in a device and method, but before this device and method have been activated to adjust airflow characteristics to reduce snoring. Accordingly, in FIG. 3 the person is still snoring. Snoring is represented by the vibration of soft tissue 102, the vibration of nasal inflow 107, and the vibration of oral inflow 108.

The example of the invention that is shown in FIG. 3 before adjustment of airflow characteristics includes: an airflow-constraining member 301 (a mouth-covering mask in this example); an airflow channel 302 that passes through this airflow-constraining member; two inflatable members, 303 and 304, that are in fluid communication with this airflow channel; and an inflation-adjusting mechanism 305 that enables selective inflation of the inflatable members. Expressing this in another way, FIG. 3 shows an example of a device to reduce snoring that comprises: an airflow-constraining member, an airflow channel or opening through this member, and an airflow-adjusting mechanism. In this example, the airflow-adjusting mechanism comprises two inflatable members, 303 and 304, in combination with an inflation-adjusting mechanism 305.

FIG. 4 shows the same device in a lateral cross-sectional view of this person's head, but after this device has been used to adjust airflow characteristics in order to reduce or prevent snoring. In this example, activation of the adjustment of airflow characteristics to reduce snoring has been done manually. Manual adjustment is represented, in this diagram, by a person's hand 306 pressing inflation-adjusting mechanism 305.

In an example, manual adjustment of a device to reduce snoring may be done by the person wearing the device, prior to sleep, in an iterative manner over the span of several nights. Each night the person tries the device at a different adjustment setting and evaluates the results or has a bed partner evaluate the results. Adjustment is done in an iterative manner to find the settings that optimally reduce snoring. In another example, manual adjustment may be done by a person other than the person wearing the device. Manual adjustment of the device may be done by a bed partner (such as during regular sleep) or by a health care professional (such as during a sleep study). Adjustment of the device by a bed partner or health care professional can be done, in real time, while the person wearing the mask is sleeping.

As we will discuss more in subsequent figures, adjustment of the device during sleep by a bed partner or by a health care professional may be preferably accomplished with the aid of a wireless remote control device. Use of a wireless remote control may be less likely to disturb the sleeping person wearing the device. Such airflow adjustments would be limited to certain ranges by safety parameters in order to ensure sufficient oxygen for the sleeping person. Although there is no direct contact between the device and a human hand when a wireless remote control is used, we still classify this as manual adjustment because the adjustment is done by direct human action, not automatically by a machine. There is no guarantee that a device that allows a bed partner to remotely adjust airflow characteristics to reduce snoring will restore relational bliss for couples whose relationships have been taxed by chronic snoring, but such a device is far preferable to having the bed partner nudge or jab the snoring person repeatedly throughout the night!

In an example, manual adjustment may comprise selectively inflating or deflating inflatable members 303 and 304 in a manner similar to manual inflation of air pockets in athletic shoes. In an example, inflation-adjusting mechanism 305 may be entirely pneumatic and not require any electrical power. In another example, an inflation-adjusting mechanism may include electrical components, such as a small-scale air compressor and/or an electronic control chip. In the latter case, the inflation-adjusting mechanism would also include a battery or some other type of electrical power source.

In the example shown in FIG. 4, the inflation or deflation of inflatable members 303 and 304 changes the cross-sectional size of airflow channel 302 and, as a result, changes the airflow characteristics of oral inflow during inhalation. The more that members 303 and 304 are inflated, the narrower airflow channel 302 becomes. In this example, inflatable members 303 and 304 are inflated in a symmetric manner In other examples, these two members may be differentially inflated to change the shape, as well as the width, of the airflow channel. In an example, the cross-sectional size of the airflow channel may be narrowed to reduce, or block entirely, air inflow through the mouth during inhalation in order to reduce or prevent the vibration of soft tissue that causes snoring. In an example, the cross-sectional size of the airflow channel may be changed to adjust airflow characteristics of oral inflow relative to the airflow characteristics of nasal inflow in order to reduce or prevent vibration of soft tissue that causes snoring.

In an example, manual adjustment of the inflation levels of inflatable members 303 and 304, to change the cross-sectional size of the airflow channel 302, can be done in an iterative manner in order to adjust the airflow characteristics of oral inflow so that the combined aerodynamics of nasal inflow 107 and oral 108 no longer cause soft tissue 102 to vibrate at its resonant frequency. This reduces or prevents snoring.

This example of a device and method to reduce snoring includes an airflow-adjusting mechanism comprising inflatable members 303 and 304 in combination with inflation-adjusting mechanism 305. The airflow-adjusting mechanism in this example enables adjustment of one or more characteristics of airflow during inhalation in order to reduce, cancel, disrupt, or prevent the vibration of soft tissue along the upper airway that causes snoring. In various examples, an airflow-adjusting mechanism may enable adjustment of one or more airflow characteristics of airflow through an airflow channel during inhalation in order to reduce or prevent vibration of soft tissue at a resonant frequency along the person's airway that causes snoring.

In this example, an airflow-adjusting mechanism enables adjustment of one or more characteristics of airflow through the airflow channel. In various examples, this airflow-adjusting mechanism may be inside this airflow channel or otherwise in fluid communication with this airflow channel. In an example, this airflow-adjusting mechanism does not extend more than twelve inches from the person's body, apart from an optional wireless (or other type of) remote control or monitoring unit. In an example, the entire invention may be self-contained within a space no more than twelve inches away from the person's body, apart from any optional remote control or monitoring units. In an example, an airflow-resisting member, an airflow channel or opening, and an airflow-adjusting mechanism can comprise a self-contained device that does not extend more than twelve inches away from the person's body. In an example, this invention may comprise a self-contained, snore-cancelling device that is worn on the person's head or located entirely in close proximity to the person's head.

One way to treat snoring is the use of Continuous Positive Airway Pressure (CPAP). However, many people do not tolerate CPAP, especially the use of a mask, being tethered to a bedside air pump, and the noise of the pump. Also, many people around the world cannot use CPAP because they lack a dependable source of electrical power that CPAP requires. The device and method disclosed herein can provide these people with an alternative to CPAP for reducing snoring. Unlike a Continuous Positive Airway Pressure (CPAP) system with a bedside air pump, the device and method for reducing snoring that are shown herein do not require that a sleeping person be tethered by an air tube to a bedside pump. Compared to most Continuous Positive Airway Pressure (CPAP) systems, the device and method for reducing snoring that are shown herein require much less energy and can be energy self-sufficient.

In an example, the operation of an airflow-adjusting mechanism, such as the one shown in these figures, does not require Continuous Positive Airway Pressure (CPAP) as an airflow input. In an example, airflow entering the airflow channel in this device during inhalation comes from ambient air, unamplified from atmospheric pressure. In an example, airflow entering this airflow channel during inhalation need not be pressurized at a level higher than the pressure of ambient air. In an example, operation of the airflow-adjusting mechanism also need not produce Continuous Positive Airway Pressure (CPAP) as an airflow output.

In an example, operation of an airflow-adjusting mechanism can reduce snoring without having to provide Continuous Positive Airway Pressure (CPAP). This can help to make this device and method self-contained, portable, and energy self-sufficient. In an example, the airflow-adjusting mechanism need not be part of system that provides Continuous Positive Airway Pressure (CPAP). In an example, operation of the air-adjusting member may provide Expiratory Positive Airway Pressure (EPAP) in an energy self-sufficient manner, but need not provide Continuous Positive Airway Pressure (CPAP). In an example, this invention can comprise a self-contained device and method for reducing snoring that does not require connection via an air-tube to a remote (e.g. bedside) CPAP air pump. This device and method can be a self-contained, portable, energy self-sufficient alternative to CPAP for treating snoring.

In an example, an airflow-adjusting mechanism can operate without a source of positive air pressure or a positive air pressure mechanism, with the possible exception of air pressure from human lungs, wherein positive air pressure is defined as air pressure that is above ambient air pressure. In an example, this invention does not require a source of positive air pressure greater than that of ambient air, other than positive air pressure from human lungs. In an example, an airflow-adjusting mechanism need not require an external source of pressurized gas or a remote air pump.

In this example, the airflow-constraining member 301 is a mask that covers the mouth only. In another example, airflow-constraining member 301 can be a mask that covers both the nose and mouth. In another example, airflow-constraining member 301 can be a mouth insert that is inserted into the mouth, instead of mouth mask that covers the mouth. In other examples, there may be more than one airflow channel through an airflow-constraining member. In other examples, there may be airflow channels that are in fluid communication with the airflow-constraining member and go around the airflow-constraining member, instead of running through it.

In an example, this airflow-constraining member may be held against the person's mouth and face by means of elastic straps. In another example, this airflow-constraining member may be held against the person's mouth and face by engagement with the person's teeth. In another example, this airflow-constraining member may be held against the person's head by adhesion. In another example, this airflow-constraining member may be held against the person's mouth and face by means of pressure from a cantilevered member. There are many different means in the prior art by which masks, or other airflow-constraining members, can be held against a person's mouth, nose, and/or face and the precise means is not central to this invention. Accordingly, the precise means is not shown here.

In various examples, an airflow-adjusting mechanism can cause one or more changes selected from the group consisting of: an increase or decrease in the size of an aperture through which air flows through the mouth; a change in the shape of an airflow channel through which air flows through the mouth; and an increase or decrease in the degree of blockage caused by a member protruding into an airflow channel through which air flows through the mouth. In an example, changes in airflow through the mouth may be caused by inflation or deflation of an inflatable member that is in fluid communication with air flowing through the mouth. In an example, rapid deflation of an inflatable member may discharge a pulse of air into the mouth that may disrupt fluttering and/or resonance of the soft tissue, thereby stopping snoring.

FIGS. 3-4 show an example of this invention wherein an airflow-constraining member that directs, reduces, or blocks respiratory airflow during sleep is a mask that covers a person's mouth. In this example, this airflow-constraining member is worn by a sleeping person. In various examples, an airflow-constraining member that directs, reduces, or blocks respiratory airflow during sleep may be selected from the group consisting of: a member inserted into a person's mouth; one or more members inserted into a person's nose; one or more members inserted into a person's mouth and nose; a mask covering a person's mouth; a mask covering a person's nose; and a mask covering both a person's mouth and nose.

In various examples, an airflow-constraining member may constrain or direct airflow by a means selected from the group consisting of: covering the mouth, covering the nose, covering both the mouth and nose, insertion of a device into the mouth, insertion of a device within the nose, and insertion of devices within both the mouth and nose. In various examples, this invention may include an airflow-constraining member that covers, blocks, channels, or directs airflow through the mouth and/or nose. In various examples, this airflow-constraining member may comprise a helmet, an air mask, a mouth insert, a dental appliance, nasal pillows, and nasal inserts. In various examples, this invention may comprise an airflow-constraining member or an airflow-channeling member that that is worn over, or inserted within, the mouth, the nose, or both the mouth and nose.

FIGS. 3-4 show an example of this invention that includes an airflow channel that allows airflow through an airflow-constraining member. In various examples, an airflow channel may be an airflow opening through an airflow-constraining member, such as an opening in a mask, mouth insert, or nasal insert. In an example, an airflow channel may allow airflow to pass through the airflow-constraining member and thereby enter, exit, or enter and exit the mouth, the nose, or the nose and the mouth. In various examples, one or more airflow channels may be in fluid communication with the airflow-constraining member even if they do not go directly through the airflow-constraining member. In various examples, there may be separate airflow channels for air inflow during inhalation and air outflow during exhalation.

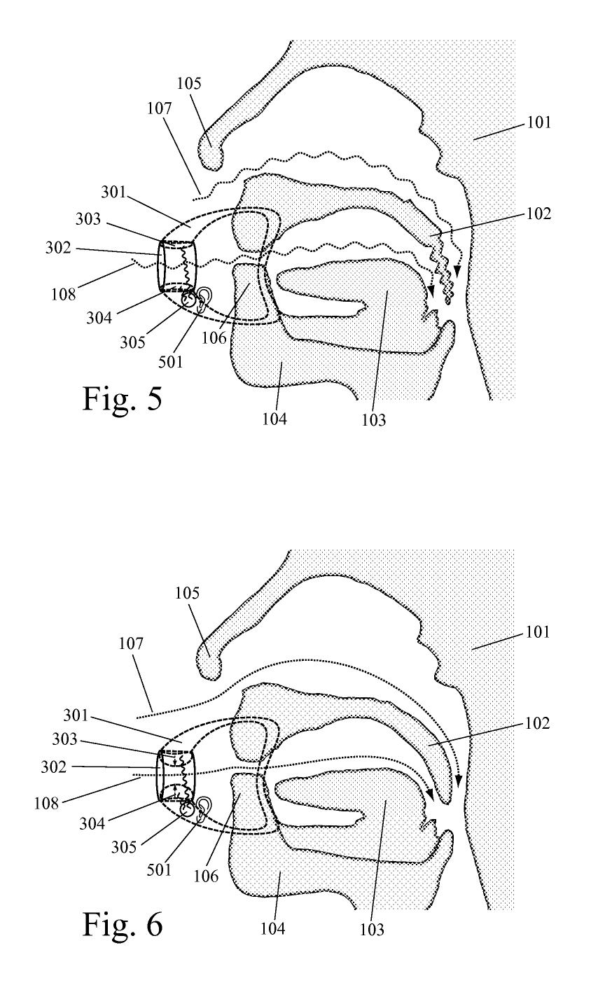

FIGS. 5-6 show another example of how this invention may be embodied. In this example, the airflow characteristics of oral inflow 108 are adjusted automatically by a device to reduce the vibration of soft tissue 102 that causes snoring. The components of the device in FIG. 5 are like those of the device shown in FIG. 3, except that the device in FIG. 5 now also has a sound-detecting member, 501. This sound-detecting member enables the inflation-adjusting mechanism, 305, to automatically analyze sounds and to adjust airflow characteristics accordingly in order to reduce snoring. FIG. 5 shows this device before automatic adjustment of airflow characteristics has occurred. In FIG. 5, soft tissue 102 is vibrating, nasal inflow 107 is vibratory, and oral inflow 108 is vibratory; the person is snoring.

FIG. 6 shows this example after the automatic adjustment of the airflow characteristics of oral inflow 108 by inflation-adjusting mechanism 305. As a result of this automatic adjustment, soft tissue is no longer vibrating; snoring has stopped. The chain of action to stop snoring in this example is as follows. Snoring is detected by sound-detecting member 501. This triggers inflation-adjusting mechanism 305. This inflates members 303 and 304. This narrows airflow channel 302. This adjusts the relative airflow dynamics of oral inflow 108 vs. nasal inflow 107 past soft tissue 102. This stops soft tissue 102 from vibrating at its resonant frequency. This stops the person from snoring.

In an example, automatic adjustment of airflow characteristics can occur while the person is sleeping, without disturbing them. In an example, airflow characteristics may be adjusted based on the prediction of snoring, by analysis of respiratory sounds, rather than based on detection of snoring after it occurs. In an example, sound-detecting member 501 and inflation-adjusting mechanism 305 can operate together to track the person's respiratory cycle and to evaluate when to cause prophylactic adjustment of airflow characteristics in order to prevent snoring. In an example, sound-detecting member 501 and inflation-adjusting mechanism 305 together comprise an airflow-adjusting mechanism. In an example, inflation-adjusting mechanism 305 may include electronics. In an example, inflation-adjusting mechanism 305 may include pneumatic components and not include any electronic components.

In various examples, while a person sleeps, a sound-detecting member and an inflation-adjusting mechanism can operate together to change automatically, over time, one or more characteristics of airflow through a person's mouth. In various examples, another type of airflow-adjusting mechanism may operate to change automatically, over time, one or more characteristics of airflow through a person's mouth while the person sleeps. These airflow characteristics can be selected from the group consisting of airflow speed, pressure and volume. These changes can alter the aerodynamics of air flowing past soft tissue in the airway in a manner that reduces the vibration, fluttering, and/or resonance of that soft tissue and thereby reduce snoring.

In an example, an airflow-adjusting mechanism can change over time, while a person sleeps, one or more characteristics of airflow through the mouth based on the person's snoring. In another example, an airflow-adjusting mechanism can change over time, while a person sleeps, one or more characteristics of airflow through the mouth based on the person's breathing cycle. Snoring and/or the breathing cycle can be detected by a sound-detecting member, such as a microphone. Adjustment of airflow at certain points in the person's breathing cycle may be able to completely prevent snoring from occurring.

FIGS. 3-4 showed an example of this invention wherein activation of adjustment of airflow characteristics was done by manual adjustment of airflow characteristics by a person.

FIGS. 5-6 show an example of a device and method to reduce snoring wherein airflow characteristics are adjusted automatically. Automatic adjustment of airflow characteristics enables adjustment of airflow characteristics while a person sleeps, such as in response to the detection or prediction of snoring. In various examples, activation of adjustment of airflow characteristics may be selected from the group consisting of: manual adjustment of airflow characteristics by a person; and automatic adjustment of airflow characteristics while a person sleeps in response to the detection or prediction of snoring. In an example, the airflow characteristics of airflow during inhalation can be adjusted automatically, while a person sleeps, in order to reduce or prevent vibration of soft tissue in order to reduce or prevent snoring. In various examples, an airflow-adjusting mechanism may automatically change airflow characteristics while a person sleeps based on the person's respiratory cycle, occurrence of snoring, or both.

In an example, an airflow-adjusting mechanism may enable adjustment of airflow characteristics in an iterative manner, in order to identify those specific airflow characteristics that are particularly successful in reducing or preventing snoring. In an example, a device may have a microphone that helps the device to sense the respiratory cycle, to identify snoring, or to do both, wherein information concerning the respiratory cycle, snoring, or both is used to help adjust airflow characteristics in an iterative manner to reduce or prevent snoring. In an example, a device may vary characteristics of airflow through the mouth in real time, in an iterative manner, in order to find the best combination of airflow characteristics that optimally reduces or prevents snoring. In an example, a device and method may employ a feedback process using variable movement of airflow-adjusting mechanisms in response to detection of snoring by a sound-detecting member, such as a microphone.

In an example, this invention can include an airflow-adjusting mechanism that enables adjustment of airflow characteristics in an iterative manner over the span of multiple respiratory cycles in order to select airflow characteristics that are successful in reducing or preventing snoring. In an example, this invention can enable programming different energy-harvesting and energy-using functions for different respiratory cycles based on the occurrence, or prediction, of snoring in a particular respiratory cycle.

In an example, an airflow-adjusting mechanism may: change the tension or inflation of a balloon; or change the pressure within an air chamber or balloon. In various examples, an airflow-adjusting mechanism may transduce the kinetic energy of airflow from human respiration into changes in the tension of a balloon or changes in the pressure within an air chamber. In an example, an airflow-adjusting mechanism may change the resistance to expansion that is given by an expanding member that is in fluid communication with an airflow channel. In various examples, an airflow-adjusting mechanism may adjust the airflow characteristics of inflow during inhalation by changing the inflation of a balloon or by changing the pressure within an air chamber. In various examples, an airflow-adjusting mechanism may transduce the kinetic energy of airflow during human respiration into changes in the tension or inflation of an expandable member, such as a balloon. In various examples, an airflow-adjusting mechanism may harvest or transduce the kinetic energy of airflow during human respiration into changes in the pressure within an air chamber or balloon.

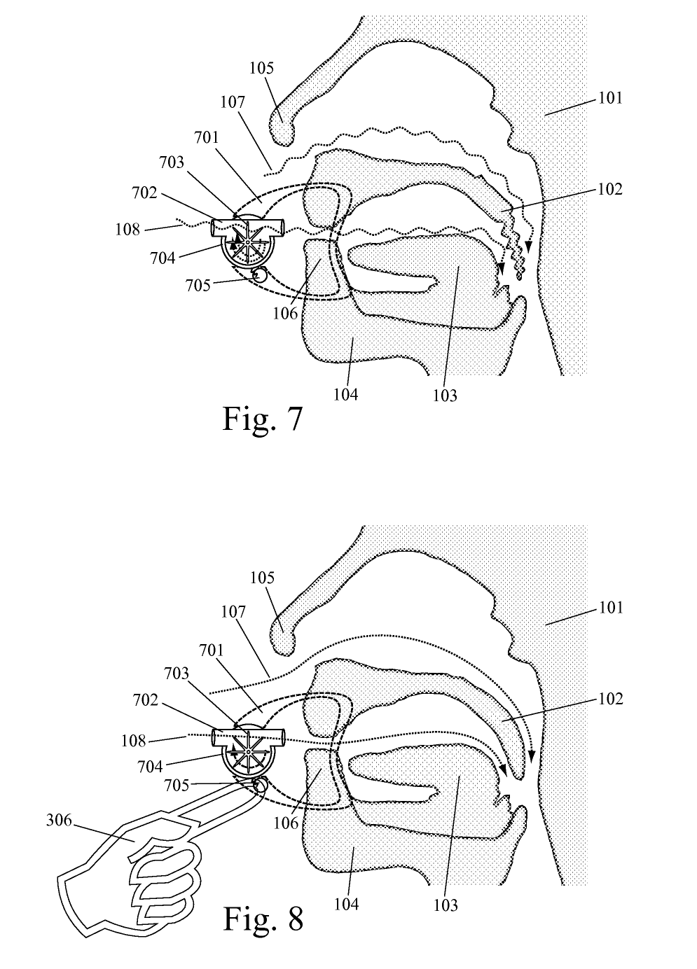

FIGS. 7-8 show another example of how this invention may be embodied in a device and method to reduce snoring. This example is like the example in FIGS. 3-4 in that adjustment of airflow characteristics to reduce snoring is also done manually. However, this example differs in the mechanism that is used to adjust airflow characteristics. In the example shown in FIGS. 7-8, a rotating impeller is used to adjust the airflow characteristics of oral inflow 108 relative to the airflow characteristics of nasal inflow 107. FIG. 7 shows this example before the airflow characteristics of oral inflow 108 have been manually adjusted, wherein the person is snoring. FIG. 8 shows this example after the airflow characteristics of oral inflow 108 have been manually adjusted using this device and method, wherein snoring has stopped.

We now discuss the example shown in FIGS. 7-8 in more detail. FIG. 7 shows an example of this invention comprising: an air-constraining member 701 (a mouth-covering mask in this example); an airflow channel 702 through the air-constraining member; an air impeller 703 that rotates within the airflow channel; a rotation-controlling member 704 that is connected to the impeller by its central axle and that adjusts or otherwise controls the rotation of the impeller; and a user interface 705 that controls the operation of the impeller via the rotation-controlling member. In this example, impeller 703, rotation-controlling member 704, and user interface 705 together comprise an airflow-adjusting mechanism that adjusts the airflow characteristics of oral inflow 108 to reduce the vibration of soft tissue 102 and thereby reduce snoring.

In an example, rotation-controlling member 704 can be a passive component that can variably and adjustably resist the rotation of impeller 703, but cannot actively rotate impeller 703. Such a passive rotational-resistance mechanism has advantages. It can be relatively simple and does not require a power source other than the direct kinetic energy of airflow from human respiration.

In another example, rotation-controlling member 704 can be an active, energy-powered component that actively drives the rotation of impeller 703. Actively-powered rotation of impeller 703 by rotation-controlling member 704 requires a power source. Active rotation can enable a greater range of airflow characteristic adjustments, including intermittent positive pressure, in order to reduce snoring. It can be possible to achieve device portability and energy self-sufficiency, even with active rotation. As we will discuss later, it is possible to power active rotation from energy harvested from human respiration, using certain embodiments of this invention. In an example, rotation-controlling member 704 may function as an actuator or motor. In an example, rotation-controlling member 704 may function as an electrical generator during one phase of respiration (such as exhalation) and function as an actuator or motor during another phase of respiration (such as inhalation).

In FIG. 7, impeller 703 rotates at a first rotational speed during inhalation. At this first rotational speed, the airflow characteristics of oral inflow 108 relative to the airflow characteristics of nasal inflow 107 cause soft tissue 102 to vibrate at a resonant frequency. Accordingly, the person snores. In FIG. 8, impeller 703 rotates at a second rotational speed during inhalation due to intervention by this device. At this second rotational speed, the airflow characteristics of oral inflow 108 interact with those of nasal inflow 107 and/or the characteristics of soft tissue 102 and eliminate vibration of soft tissue 102 at a resonant frequency. Accordingly, snoring stops.

In this example, the second rotational speed is slower than the first rotational speed, as indicated by a change from two dotted motion-indicating arrows in FIG. 7 to just one arrow in FIG. 8. In another example, the second speed may be faster than the first speed. One's choice of impeller rotational speed to reduce snoring depends on the aerodynamics of oral and nasal airflows as they flow past both sides of soft tissue 102 and the resonant frequency of this soft tissue.

In this example, passive rotation-controlling member 704 passively creates a slower second speed by offering greater resistance to rotation. In another example, an active rotation-controlling member may actively create a faster second speed by actively rotating the impeller. In this example, the selection of the second rotational speed for the rotating impeller is determined manually. This is indicated in this figure by person's hand 306 adjusting user interface 705. In another example, the selection of the second rotational speed of the rotating member may be determined automatically based on a feedback loop involving a sound-detecting member incorporated into the device.

FIGS. 7-8 show an example of this invention in which an airflow-adjusting mechanism adjusts airflow characteristics to reduce snoring by changing the rotational speed of an air impeller. In various examples, an airflow-adjusting mechanism may adjust airflow characteristics to reduce snoring by changing the rotational speed, resistance, momentum, or direction of some other rotating member such as a turbine, fan blade, paddlewheel, flywheel, helix, screw, or gear. In an example, an airflow-adjusting mechanism may transduce some of the kinetic energy of airflow from human respiration into the rotation of an impeller. This can help to make the device and method partially or completely energy self-sufficient. In various examples, an airflow-adjusting mechanism may transduce some of the kinetic energy of airflow from human respiration into the rotation of a rotating member such as a turbine, fan blade, impeller, paddlewheel, flywheel, helix, screw, or gear.

In various examples, an airflow-adjusting mechanism may adjust one or more dynamics of one or more rotating members that are in fluid communication with an airflow channel. These dynamics may be selected from the group consisting of: rotational speed, rotational torque or resistance, and rotational direction. In various examples of this invention, an airflow-adjusting mechanism may comprise one or more rotating members in fluid communication with an airflow channel, wherein these members are selected from the group consisting of turbine, fan blade, impeller, paddlewheel, flywheel, helix, screw, and gear.

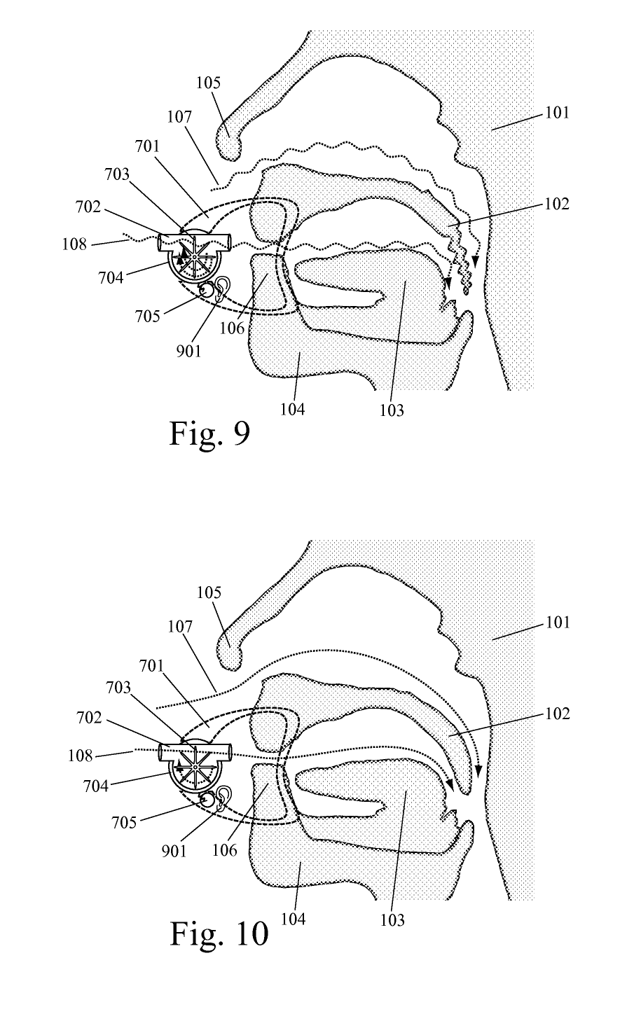

FIGS. 9-10 show an example of this invention that is like the example shown in FIGS. 7-8, except that now the adjustment of airflow characteristics is done automatically by the device employing sound-detecting member 901 (which may be a microphone and is represented symbolically by an ear symbol) in combination with user interface 705 and rotation-controlling member 704. Automatic adjustment of airflow characteristics while a person sleeps makes it easier to identify that combination of airflow characteristics that optimally reduces or prevents snoring. Automatic adjustment of airflow characteristics can be done in an iterative manner while a person sleeps. The device can adjust characteristics within a range of safe parameters and measure which characteristics optimally reduce snoring sounds. In an example, this invention enables adjustment of airflow characteristics in an iterative manner over the span of multiple respiratory cycles in order to select airflow characteristics that are successful in reducing or preventing snoring.

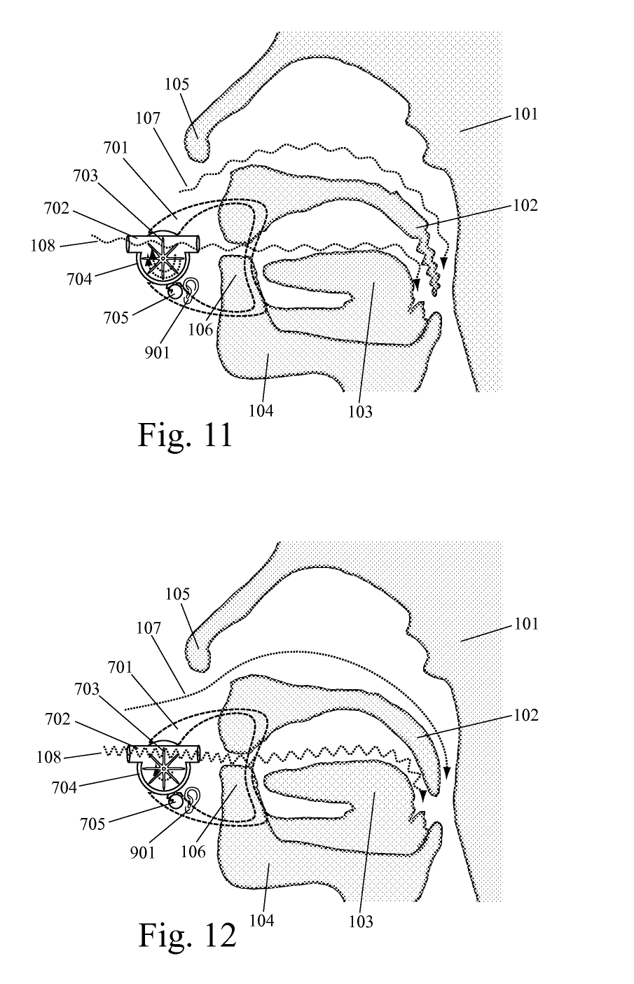

FIGS. 11-12 show an example of the invention that is similar in hardware to the example shown in FIGS. 9-10, but functions differently. In FIGS. 11-12, an airflow-adjusting mechanism (comprised of impeller 703, rotation-controlling member 704, interface 705, and sound-detecting member 901) creates a series of air pulses in the oral inflow. This series of air pulses disrupts or cancels the harmonic vibration of soft tissue 102. By analogy, air turbulence can change the lift of an airplane wing and a series of air pulses can disrupt the resonant tone of a woodwind instrument.

In an example, the blades of rotating impeller 703 can create a series of air pulses. The frequency of the pulses is determined by the speed of the rotation of the impeller. In an example, impeller 703 can create a series of air pulses in a passive manner during inhalation, with the frequency of pulses determined by the degree of passive rotational resistance given by rotation-controlling member 704. In another example, impeller 703 can create a series of air pulses in an active manner during inhalation, with the frequency of the pulses determined by the speed of rotation of the impeller, wherein the rotation-controlling member acts as an actuator (or motor) to actively drive rotation of the impeller. In various examples, the frequency and amplitude of air pulses within airflow is affected by the size and shape of the impeller blades. For example, curved blades will cause a different pattern of air pulses than straight blades. In an example, these variables may be completely controlled in a manual manner via a user interface. In an example, these variables may be partially or entirely controlled in an automatic manner by interface 705, comprising an electronic component.

In an example, an increase or decrease in the size or frequency of the air pulses produced by one or more rotating, or oscillating, members can create a pattern of air pulses and/or sound waves that counteracts, or cancels out, the resonance of soft tissue in the airway and thereby reduces snoring. In various examples, an airflow-adjusting mechanism, such as one composed of an actuator and rotating or oscillating member, can adjust over time, while a person sleeps, one or more characteristics of airflow through the person's mouth. These characteristics may be selected from the group consisting of the timing of one or more air pulses, the size of one or more air pulses, sound frequency, sound amplitude, and sound pattern. In an example, an airflow-adjusting mechanism may create a series of air pulses during inhalation that disrupts the vibration of soft tissue along the airway at a resonant frequency.

In an example, wave energy can be imparted into airflow through the mouth, wherein this energized airflow impacts the mouth-facing side of soft tissue in a manner that cancels out the resonant wave pattern of that tissue which would otherwise cause snoring. In an example, a device may iteratively adjust characteristics of this wave energy in order to find that pattern of wave energy that optimally reduces or avoids snoring. In an example, an airflow-adjusting mechanism can create a pattern of air pulses or sound waves that counteracts or cancels out the fluttering of soft tissue in the airway and thereby reduces snoring.

In various examples, this invention may be embodied in a method for reducing snoring, comprising: changing over time, while a person sleeps, one or more characteristics of airflow through the person's mouth based on the person's breathing cycle, snoring, or both breathing cycle and detection of snoring: wherein these characteristics are selected from the group consisting of speed, pressure, volume, timing of one or more air pulses, size of one or more air pulses, sound frequency, sound amplitude, and flow direction; wherein the airflow through the mouth is selectively changed differently than airflow through the nose; and wherein these changes alter the aerodynamics of air flowing past soft tissue in the airway to reduce resonance and/or fluttering of that soft tissue and thereby reduce snoring.

In various examples, this invention may be embodied in a device that adjusts airflow characteristics of air inflow through the mouth, airflow through the nose, or airflow through the mouth and nose. In various examples, these airflow characteristics may be selected from the group consisting of: airflow speed, airflow rate, airflow pressure, airflow volume, airflow duration, airflow timing, airflow direction, the creation of one or more pulses of air, the number of air pulses, pattern of air pulses, the frequency of air pulses, the magnitude of air pulses, and the duration of air pulses.

In an example, adjusting characteristics of airflow during inhalation can create a series of pulses that disrupt or cancel out harmonic vibration of soft tissue along the airway. In an example, an airflow-adjusting mechanism that is part of this invention can create a pattern of air pulses that disrupts the harmonic resonance of soft tissue along the airway. In an example, adjusting characteristics of airflow during inhalation creates a series of pulses that disrupts or cancels out vibration of soft tissue along the airway at a resonant frequency.

In various examples, an airflow-adjusting mechanism may comprise one or more mechanisms selected from the group consisting of: changing the rotational speed, resistance, momentum, or direction of a rotating member such as a turbine, fan blade, impeller, paddlewheel, flywheel, helix, screw, or gear; changing the oscillating speed, resistance, frequency, momentum, or direction of an oscillating member such as a reed, flap, valve, ball, or speaker diaphragm; changing the shape, width, length, size, or closure of one or more airflow channels that allow airflow through the airflow-constraining member; changing the tension of an extendable or compressible member such as a spring or elastic band; changing the inflation of an expandable member such as a balloon; and changing the pressure within an air chamber.

In an example, this airflow-adjusting mechanism can adjust the relative differences between airflow characteristics of airflows through the mouth vs. nose in order to alter the combined aerodynamics of these flows on soft tissue along the airway. In an example, the airflow-adjusting mechanism can change the airflow characteristics of airflow through the mouth relative to the airflow characteristics of airflow through the nose in order to disrupt harmonic resonance of soft tissue along the airway. In an example, the airflow-adjusting mechanism selectively changes airflow through the mouth differently than airflow through the nose. In an example, the airflow-adjusting mechanism can change the airflow characteristics of airflow through the mouth, but not change the airflow characteristics of airflow through the nose.

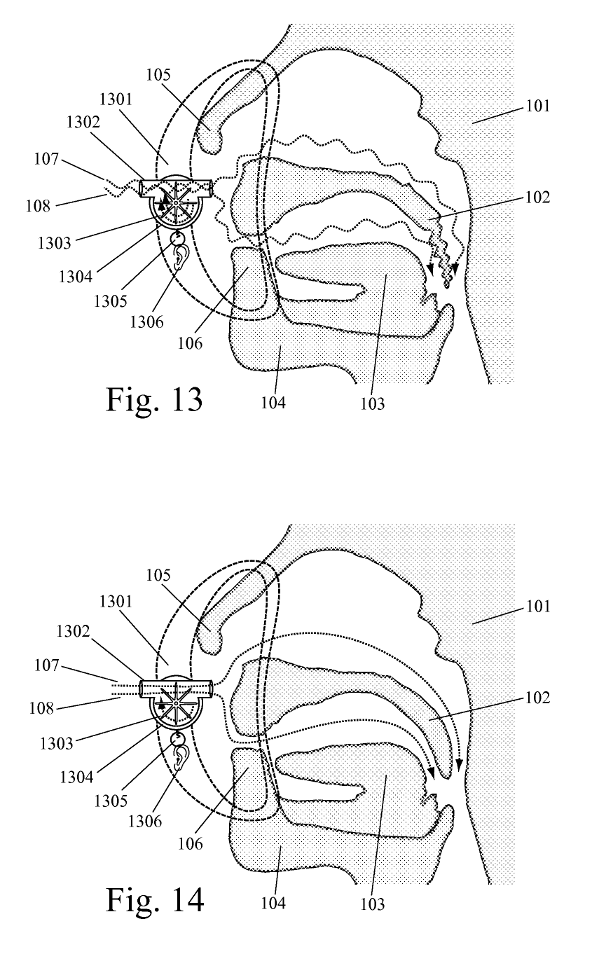

FIGS. 13-14 show another example of how this invention may be embodied. This example is similar to the example shown in FIGS. 9-10, except that now the airflow-constraining member (such as a mouth and nose covering mask) covers both the mouth and nose. The device in this example includes: airflow-constraining member 1301 (mouth and nose covering mask), airflow channel 1302 through the airflow-constraining member, air impeller 1303 within the airflow channel, impeller rotation-controlling member 1304 that is axially connected to the impeller, interface member 1305, and sound-detecting member 1306. In this example, both nasal inflow 107 and oral inflow 108 enter the interior of the airflow-constraining member 1301 through the same airflow channel 1302. In other examples, nasal and oral inflows may enter the interior of an airflow-constraining member via different airflow channels. The latter can allow more selective control and differential modification of the two inflows.

FIG. 13 shows this device before adjustment of airflow characteristics, wherein soft tissue 102 vibrates and the person snores. FIG. 14 shows this device after adjustment of airflow characteristics of both oral inflow 108 and nasal inflow 107 by the device and method, wherein the adjusted airflows no longer cause soft tissue 102 to vibrate and the person stops snoring. In this example, adjustment of airflow characteristics can occur in an automatic and iterative manner based on analysis of sound information received by the sound-detecting member 1306 and analyzed by interface member 1305.

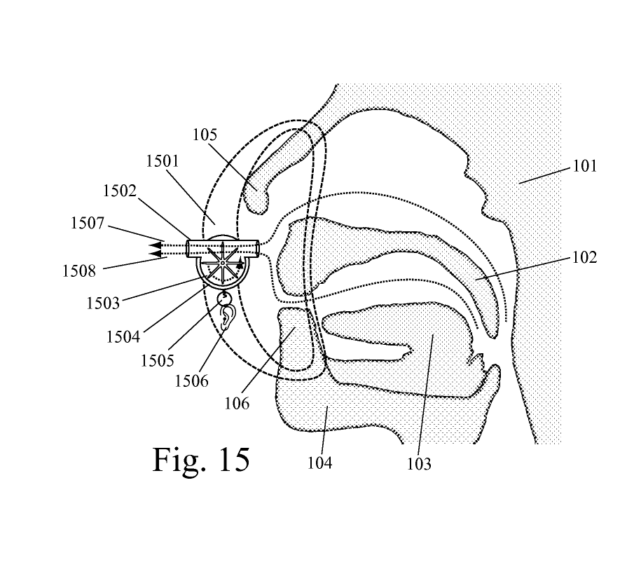

FIG. 15 shows an example of how a device similar to the one introduced in FIGS. 13-14 can be used to harvest or transduce some of the kinetic energy of airflow during exhalation. This harvested energy may be stored and used to power the device, making the device partially or completely energy self-sufficient, as well as portable and self-contained.

FIG. 15 shows a device and method to reduce snoring comprising: airflow-constraining member 1501 (a mask covering both the nose and mouth in this example); airflow channel 1502 through the airflow-constraining member; rotating impeller 1503 within the airflow channel; combination actuator/generator 1504 that is axially connect to the rotating impeller; energy-storing and controlling interface 1505; and sound-detecting member 1506.

FIG. 15 is the first of the figures herein to show airflows through the mouth and nose during exhalation, as opposed to inhalation. Nasal outflow 1507 is airflow going out through the nose during exhalation. Nasal outflow 1507 is represented by a dotted line with an end arrow pointing outward into ambient air. Oral outflow 1508 is airflow going out through the mouth during exhalation. Oral outflow 1508 is represented by a dotted line with an end arrow pointing outward into ambient air. In this example, both nasal outflow 1507 and oral outflow 1508 exit the interior of airflow-constraining member 1501 through the same airflow channel 1502. In other examples, nasal and oral outflows may exit an airflow-constraining member via different airflow channels. The latter can allow more selective control and modification of the two outflows.

In FIG. 15, the kinetic energy of the combined nasal and oral outflows, 1507 and 1508, rotates impeller 1503 during exhalation. The rotation of the central axle of impeller 1503, in turn, rotates the central shaft of multi-functional actuator/generator 1504. The multi-functional actuator/generator can: act as an actuator to transduce electrical energy into rotational kinetic energy; and act as a generator to transduce rotational kinetic energy into electrical energy. In this step of the energy-harvesting process, actuator/generator 1504 acts as a generator. Rotation of the central shaft of actuator/generator 1504, acting as a generator at this time, generates electrical energy. In this example, this electrical energy is stored in energy-storing and controlling member 1505. This stored energy can then be used later, such as during inhalation, in order to adjust the airflow characteristics of nasal and oral inflows to reduce snoring. For example, this energy may be used to increase or decrease the flow rate and/or pressure of nasal and oral inflows.

In various examples, the actuator/generator 1504 that is used as a generator to harvest energy during exhalation may be used in reverse, as an actuator, during inhalation. For example, when sound-detecting member 1506 detects (or predicts) snoring, then the device may use harvested and stored energy to accelerate inflow to provide positive air pressure and reduce snoring. For example, the device may use actuator/generator 1504 in reverse, as an actuator powered by stored energy, to rotate impeller 1503 and accelerate air inflow to reduce snoring. In another example, the device may use actuator/generator 1504 to create a series of air pulses that reduces snoring.

FIG. 15 shows an example of this invention wherein an airflow-adjusting mechanism harvests or transduces some of the kinetic energy of airflow from human respiration in order to adjust airflow characteristics to reduce the vibration of soft tissue. In this example, this invention comprises transducing the kinetic energy of human respiration to reduce the vibration of soft tissue along the airway. In an example, the airflow-adjusting mechanism may transduce or harvest the kinetic energy of airflow from human respiration into electrical energy that is used to adjust airflow characteristics to reduce vibration of soft tissue. In an example, an airflow-adjusting mechanism may transduce or harvest the kinetic energy of airflow from human respiration into another form of energy that is then used to adjust airflow characteristics to reduce vibration of soft tissue.

In an example, an airflow-adjusting mechanism can transduce or harvest the kinetic energy of human inhalation in order to create a series of air pulses that reduces the vibration of soft tissue along the airway. In an example, an airflow-adjusting mechanism can transduce or harvest the kinetic energy of human exhalation in order to create a series of air pulses that reduces the vibration of soft tissue along the airway. In an example, an airflow-adjusting mechanism can transduce or harvest the kinetic energy of human exhalation over the span of multiple respiratory cycles before the energy is used to reduce the vibration of soft tissue along the airway.

In an example, this invention may not require any external power source or any external source of pressurized air, due to its transducing or harvesting energy from human respiration. In an example, this invention may not require a source of power other than the kinetic energy of human respiration. In various examples of this invention, an airflow-adjusting mechanism may be powered, in whole or in part, by harvesting, or otherwise transducing, some of the kinetic energy of airflow from human respiration. In an example, an airflow-adjusting mechanism may harvest energy from exhalation and use this energy to adjust characteristics of airflow during inhalation to reduce or prevent vibration of soft tissue along the airway in order to reduce or prevent snoring. In an example, an airflow-adjusting mechanism may harvest energy from exhalation, accumulate and store this energy over multiple respiratory cycles, and then use this energy to adjust characteristics of airflow during inhalation to reduce or prevent snoring during a particular respiratory cycle.

In various examples, an airflow-adjusting mechanism may be powered by one or more means selected from the group consisting of: electrical energy; non-electrical energy transduced from the respiration of the sleeping person in real time; stored energy that has been previously transduced from the respiration of the sleeping person; pneumatic energy; hydraulic energy; and energy transduced from an electromagnetic field.

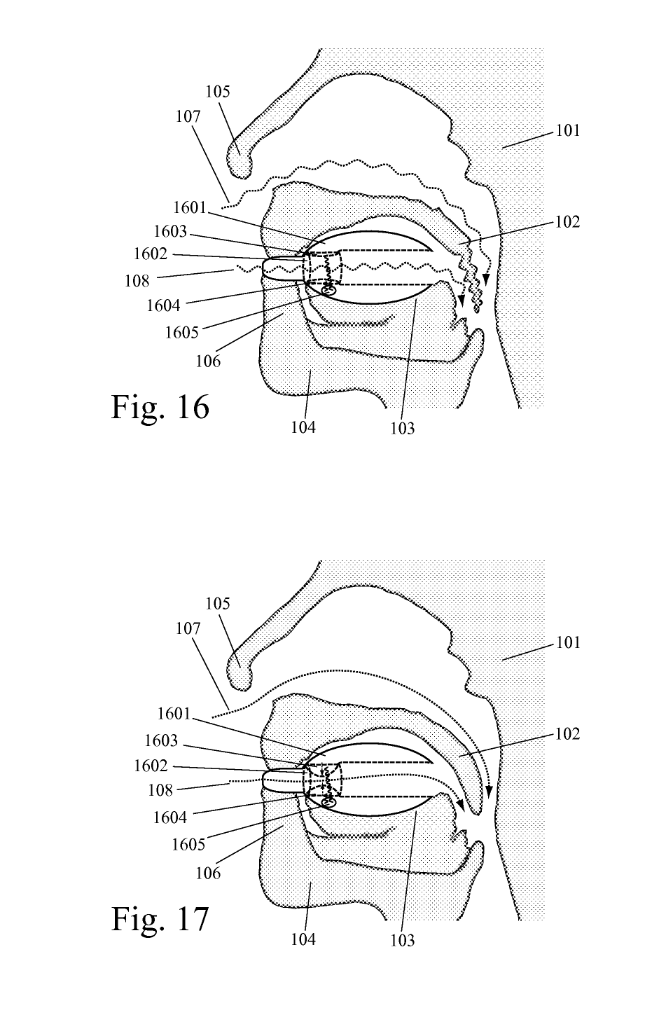

FIGS. 16-17 show another example of how this invention may be embodied. This example is similar to the example that was shown in FIGS. 5-6 in that it includes automatic adjustment of the airflow characteristics of oral inflow via adjustment of inflatable members within an airflow channel. However, the example shown in FIGS. 16-17 is embodied in a mouth insert rather than mouth-covering mask. This embodiment may be more acceptable to people who cannot tolerate having a mask press against their face while they sleep. However, it does require that the components be more compact. FIG. 16 shows this device and method comprising: an airflow-constraining member (mouth insert or dental appliance in this example) 1601; an airflow channel 1602 through the airflow-constraining member; inflatable members 1603 and 1604 within the airflow channel; and inflation controller 1605.

FIG. 16 shows this device before the adjustment of airflow characteristics by the device and method, wherein soft tissue 102 vibrates and the person is snoring. FIG. 17 shows this device after partial inflation of inflatable members 1603 and 1604, which reduces oral inflow 108, which stops the vibration of soft tissue 102, which stops the person from snoring. In this example, the mouth insert is fully inserted into the person's mouth. In an example, a mouth insert may be only partially inserted into the mouth. In an example, a mouth insert may be connected to an external mask. In an example, a mouth insert may have a bulbous portion that is inserted into the mouth, a bulbous portion that remains outside the mouth, and a thin connecting portion that connects the two bulbous portions. In an example, the mouth insert can be held in place by engagement with the person's teeth. There are many means in the prior art for holding a mouth insert or dental appliance in place and the precise means is not central to this invention.

In various examples, this invention may comprise a device that reduces snoring, comprising: a mouth insert or dental appliance that directs airflow through the mouth; and an airflow-adjusting mechanism that is in communication with the mouth insert: wherein this airflow-adjusting mechanism changes over time, while a person sleeps, one or more characteristics of airflow through the person's mouth; wherein these characteristics are selected from the group consisting of speed, pressure, volume, timing of one or more air pulses, size of one or more air pulses, sound frequency, sound amplitude, and flow direction; and wherein these changes alter the aerodynamics of air flowing past soft tissue in the airway to reduce resonance or fluttering of that soft tissue and thereby reduce snoring.

In various examples, this invention may include an airflow-adjusting mechanism that causes one or more changes selected from the group consisting of: an increase or decrease in the size of an aperture through which air flows through the mouth; a change in the shape of an airflow channel through which air flows through the mouth; an increase or decrease in the degree of blockage caused by a member protruding into an airflow channel through which air flows through the mouth; an increase or decrease in the rate of rotation of a rotating member that impels air flowing through the mouth; a change in the direction of rotation of a rotating member that impels air flowing through the mouth; an increase or decrease in the size or frequency of oscillation of an oscillating member that is in fluid communication with air flowing through the mouth; and inflation or deflation of an inflatable member that is in fluid communication with air flowing through the mouth.

These figures show an example of how this invention may be embodied in a device and method that reduces or prevents snoring. In an example, this invention includes a mouth insert that directs airflow through the mouth and an airflow-adjusting mechanism that is in fluid communication with the mouth insert. The airflow-adjusting mechanism can change over time, while a person sleeps, one or more characteristics of airflow through the person's mouth. These characteristics may be selected from the group consisting of speed, pressure, volume, timing of one or more air pulses, size of one or more air pulses, sound frequency, sound amplitude, and flow direction. Changes in these characteristics alter the aerodynamics of air flowing past soft tissue in the airway in order to reduce resonance or fluttering of that soft tissue and thereby reduce snoring.

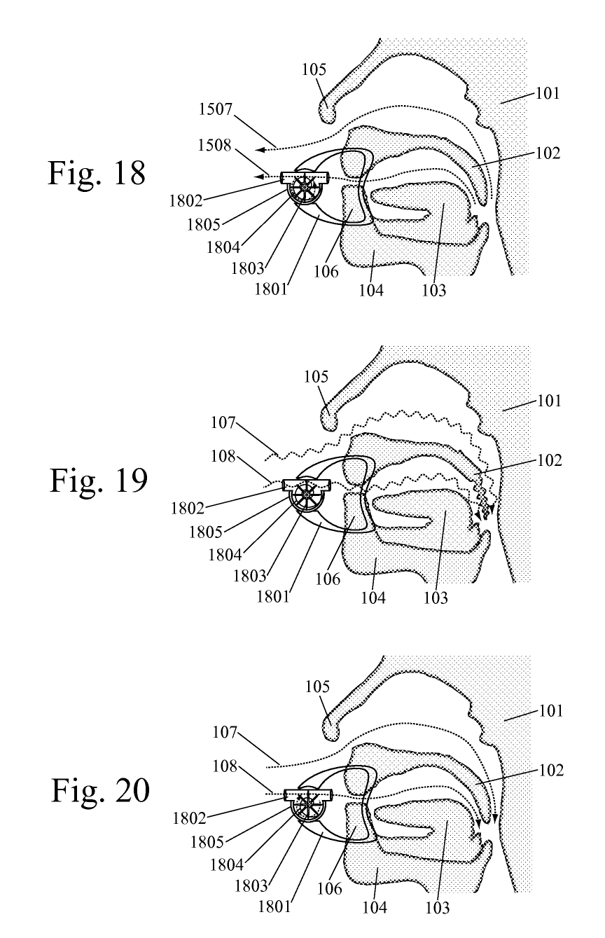

FIGS. 18-20 show another example of how this invention may be embodied in a device and method to reduce or prevent snoring. This example shows how some of the kinetic energy of air outflow during exhalation may be harvested and stored in the tensile energy of a coiled spring. Then, during inhalation, this energy may be selectively released to adjust air inflow in order to reduce snoring. This embodiment of the invention can be completely portable, self-contained, and energy self-sufficient.

FIG. 18 shows this device comprising: an airflow-constraining member 1801 (a mouth-covering mask in this example); an airflow channel 1802 through the airflow-constraining member; an air impeller 1803 within the airflow channel; a spiral coil spring 1804 that is axially-connected to the impeller; and rotation control member 1805 which is also axially-connected to the impeller. FIG. 18 shows this device during exhalation. FIG. 19 shows this device during inhalation, without adjustment of airflow characteristics, wherein the person is snoring. FIG. 20 shows this device during inhalation, with adjustment of airflow characteristics, wherein the person is not snoring.

In FIG. 18, the kinetic energy of oral outflow 1508 moving through air channel 1802 rotates impeller 1803 counter-clockwise. This rotation winds up spiral coil spring 1803. In this manner, the device transduces some of the kinetic energy from air outflow into tensile energy stored in the coiled spring. In FIG. 20, this tensile energy is released during inhalation as spiral coil spring 1803 unwinds, rotating impeller clockwise and increasing oral inflow 108. In this example, increased oral inflow 108 provides sufficient positive pressure at the onset of inhalation to reduce or prevent the vibration of soft tissue 102 and thereby reduce or prevent snoring. In this example, nasal outflow 1507 does not pass through airflow-constraining member 1801. In another example, the airflow-constraining member can be a mask that covers the nose as well as the mouth, in which case both oral and nasal outflows would pass through the airflow-constraining member.

In an example, rotation control member 1805 can be designed to create a delay between when spiral spring coil 1803 is wound up during exhalation and when the spiral spring coil 1803 begins to unwind. This delay may be timed to release the energy that was harvested during exhalation at the onset of inhalation after a lag. In other examples, rotational control member 1805 may include electronics that enable more complicated control of energy harvesting and release over multiple respiratory cycles. For example, the spiral coil spring 1803 may be incrementally and cumulatively wound up during the course of multiple respiratory cycles and this energy is not released until triggered by detection of snoring. In an example, spiral coil spring may accumulate energy from exhalation over 10 respiratory cycles before releasing this energy to accelerate inhalation during a particular respiratory cycle when snoring is detected or predicted.

In an example, there may be a series of gears between the rotation of impeller 1803 and the coiling of spiral spring 1804. This can allow incremental and cumulative harvesting of energy over multiple respiratory cycles and provide more powerful and prolonged acceleration of inhalation when needed during a particular respiratory cycle. In an example, there can be an assembly of gears that enables harvesting a small amount of energy from exhalation in each respiratory cycle, but accumulates energy over multiple respiratory cycles to provide a relatively powerful pulse of air when needed during inhalation in a selected respiratory cycle. In an example, this series of gears may create a gear ratio between rotation of the impeller and rotation of the coiled spring that is less than 1. In another example, this series of gears may create a gear ratio that is more than 1.

In various examples, this invention may include an airflow-adjusting mechanism that adjusts the airflow characteristics of oral inflow by changing the tension of a coiled spring. In an example, the airflow-adjusting mechanism may transduce the kinetic energy of airflow from human respiration into changes in the tension of an extendable or compressible member such as a spring or elastic band. In various examples, the airflow-adjusting mechanism may adjust airflow characteristics by changing the tension of an extendable or compressible member such as a spring or elastic band. In an example, a coiled spring may be wound by a clockwork mechanism that is powered by oscillating motion created by airflow, instead of being wound by the rotation of a rotating member driven by airflow.

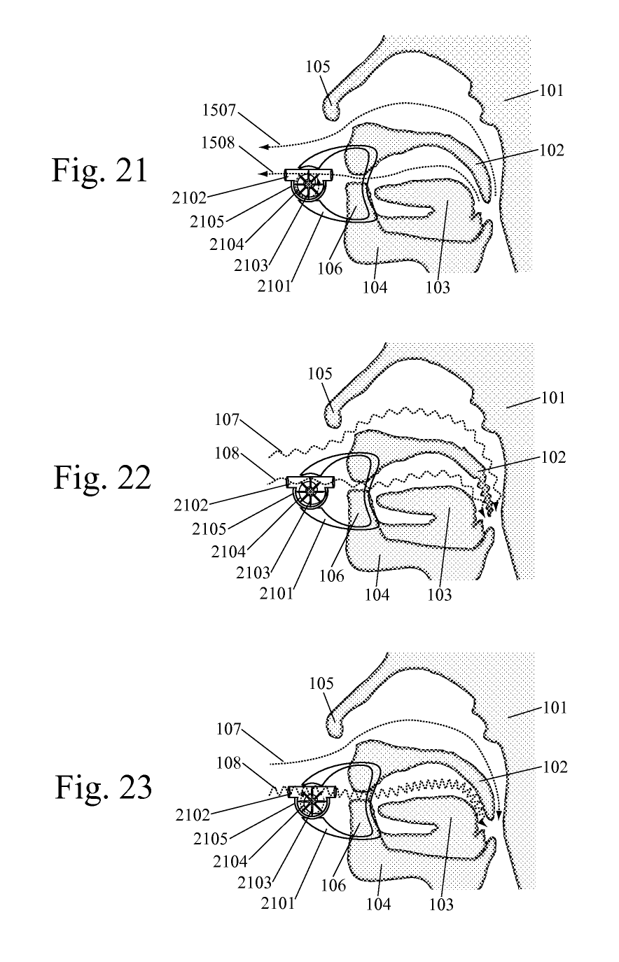

FIGS. 21-23 show an example of this invention that is similar in hardware to the example shown in FIGS. 18-20, but one which creates a series of air pulses during inhalation in order to reduce snoring. In this example, this series of air pulses acts to disrupt or cancel the vibration of soft tissue 102 at its resonant frequency. This reduces or prevents snoring. FIG. 21 shows this device comprising: an airflow-constraining member 2101 (a mouth-covering mask in this example); an airflow channel 2102 through the airflow-constraining member; an air impeller 2103 within the airflow channel; a spiral coil spring 2104 that is axially-connected to the impeller; and a rotation control member 2105 which is also axially-connected to the impeller.

FIG. 21 shows this device during exhalation wherein the device is harvesting or transducing some of the kinetic energy of airflow during exhalation. FIG. 22 shows this device during inhalation without adjustment of airflow characteristics, wherein the person is snoring. FIG. 23 shows this device during inhalation wherein oral airflow has been adjusted to comprise a series of air pulses that disrupts the vibration of soft tissue at a resonant frequency and thereby stops the person from snoring.

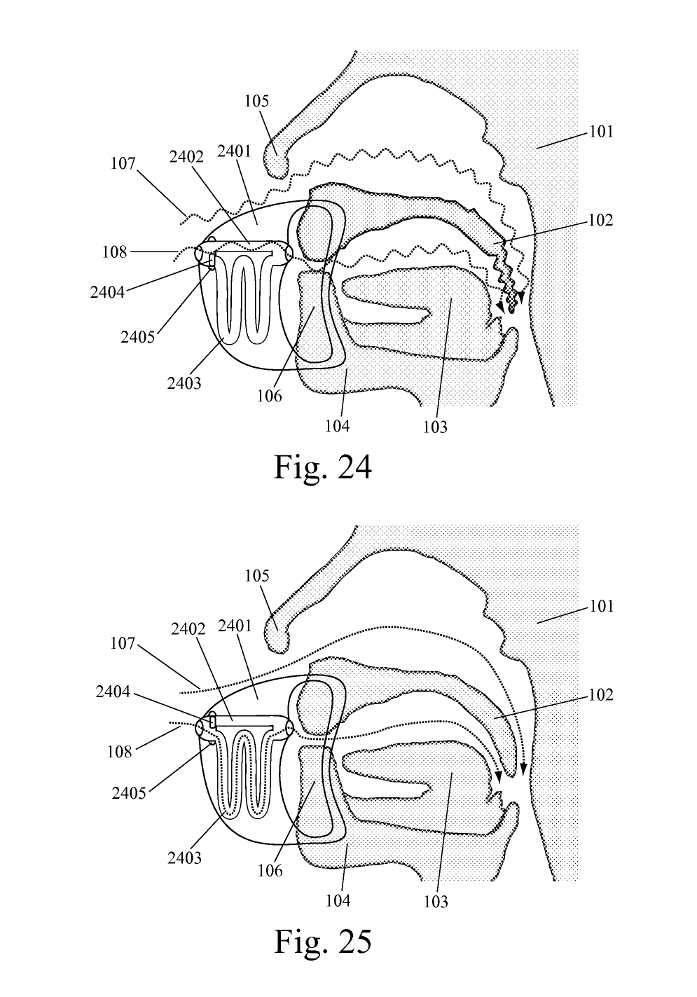

FIGS. 24-25 show another example of how this invention may be embodied in a device and method to reduce or prevent snoring. This example shows how changes in the length, size, and/or shape of an airflow channel can be changed to adjust airflow characteristics in order to reduce or prevent snoring. FIG. 24 shows this device comprising: an airflow-constraining member 2401 (a mouth-covering mask in this example); a first (short and straight) airflow channel 2402 through the airflow-constraining member; a second (longer and sinusoidal) airflow channel 2403 through the airflow-constraining member; a sliding air valve 2404 that selectively closes either the first airflow channel or the second airflow channel; and a valve-controlling mechanism 2405 that controls the position of the sliding air valve.

FIG. 24 shows this device before adjustment of airflow characteristics, wherein soft tissue 102 is vibrating and the person is snoring. FIG. 25 shows this device after adjustment of the airflow characteristics of oral inflow 108 by the device and method, wherein soft tissue 102 is no longer vibrating and the person is no longer snoring. In this example, the airflow characteristics of oral inflow 108 are changed by changing the length and shape of the airflow channel through which the oral inflow passes. In an example, adjustment of the location of sliding air valve 2404 via valve-controlling mechanism 2405 diverts oral inflow 108 from the first (short and straight) airflow channel 2402 to the second (longer and sinusoidal) airflow channel 2403. Differentially diverting airflow in this manner can change the oral airflow rate and pressure so as to disrupt the vibration of soft tissue 102 at its resonant frequency, thereby reducing or preventing snoring. In various examples, valve-controlling mechanism 2405 can be activated manually or automatically.

In an example, in addition to adjusting airflow characteristics on the way in, the longer sinusoidal air channel may also dampen the transmission of snoring sounds coming out of the mouth. By analogy, the long sinusoidal air channel may serve as a selectively activated or adjusted snoring sound muffler. In this example, the airflow-constraining member with multiple, different-shape airflow channels only covers the person's mouth. In another example, an airflow-constraining member with multiple, different-shape airflow channels may cover both the person's mouth and nose. However, in this latter case it is important that airflow resistance during inhalation not be overly constrained.

In various examples, this invention can comprise an airflow-adjusting mechanism that adjusts airflow characteristics and reduces snoring by changing the closure of one or more airflow channels that allow airflow through the airflow-constraining member. In various examples, the airflow-adjusting mechanism can change the shape, width, length, size, or closure of one or more airflow channels that allow airflow through the airflow-constraining member. In various examples, the airflow-adjusting mechanism can transduce the kinetic energy of airflow from human respiration into changes in the shape, width, length, size, or closure of one or more airflow channels that allow airflow through the airflow-constraining member.

In various examples, the mechanism of airflow adjustment may be selected from a change in one or more factors in the group consisting of: airflow channel size; airflow channel length; airflow channel shape; and the airflow channel through which flow is directed when there are multiple airflow channels. In various examples, an airflow-adjusting mechanism may change one or more characteristics of an airflow channel or opening that are selected from the group consisting of: channel or opening shape, channel or opening width, channel or opening length, and closure or opening or one or more air valves that are in fluid communication with the airflow channel or opening.

In the example shown in FIGS. 24-25, there are two different airflow channels, each with a fixed length and size. Airflow characteristics are changed by diverting oral inflow either through the first airflow channel or through the second airflow channel. In another example, the lengths of one or more airflow channels may be changed by an actuator, in a manner analogous to changes in airflow length through a trombone from changes in extension of the trombone slide. The latter example allows more gradual and precise control of airflow characteristics during airflow adjustment in order to optimally reduce soft tissue vibration.

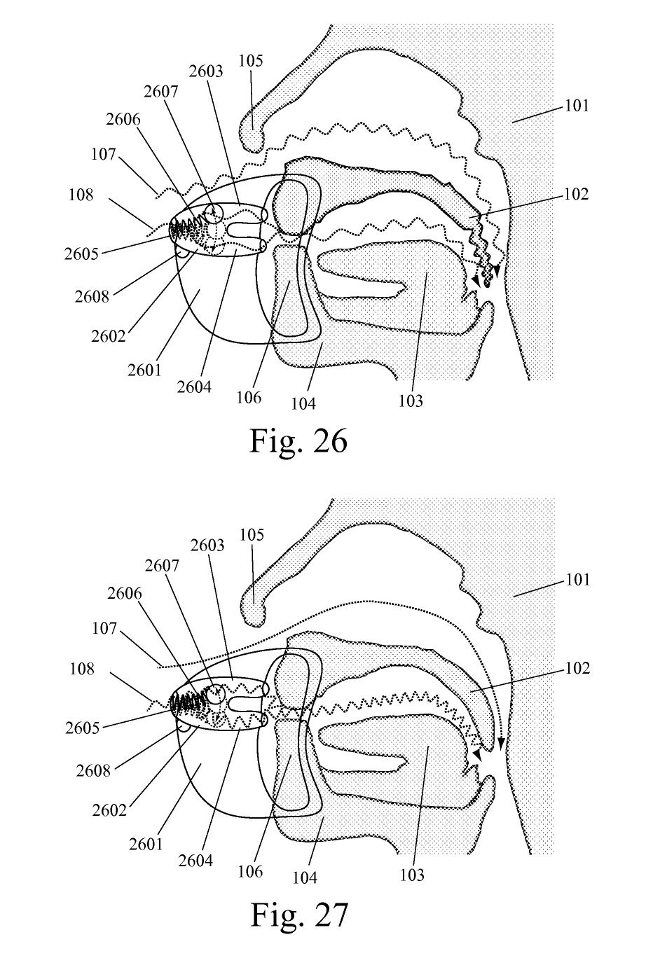

FIGS. 26-27 show another example of this invention embodied in a device and method to reduce snoring. This example uses an oscillating member to create a series of air pulses in airflow during inhalation, wherein this series of air pulses disrupts or cancels vibration of soft tissue at a resonant frequency and stops snoring. FIG. 26 shows this device before airflow adjustment, wherein the person is snoring. FIG. 27 shows this device after airflow adjustment, wherein the person is no longer snoring.

FIG. 26 shows this device comprising: an airflow-constraining member 2601 (a mouth-covering mask in this example); a U-shaped airflow channel 2602 through the airflow-constraining member, with upper airflow branch 2603 and lower airflow branch 2604; an opening 2605 from ambient air into this U-shaped airflow channel to allow ambient air to enter the airflow channel; an oscillating ball 2607 within the airflow channel and a spring 2606 with one end attached to the ball and the other end attached to the front of the airflow-constraining member; and a spring-adjusting mechanism 2608 that adjusts the position and/or tension of the spring.

In this example, the kinetic energy and aerodynamics of oral inflow 108 flowing through the airflow channel cause ball 2607 to oscillate between the entrances to the upper airflow branch 2603 and the lower airflow branch 2604. This causes airflow to oscillate between flowing through upper airflow branch 2603 and lower airflow branch 2604. This oscillation imparts a series of air pulses to oral inflow 108 as it passes through the mouth. Adjusting the position and/or tension of spring 2606 changes the oscillation frequency of ball 2607 in response to oral inflow. This changes the frequency of air pulses in oral inflow 108. In other examples, a similar effect may be achieved with a different type of oscillating member, such as an oscillating reed or air valve.

In this example, the chain of energy transduction is as follows. Adjusting the position and/or tension of spring 2606 changes the oscillation frequency of ball 2607. This change in oscillation frequency changes the frequency of air pulses imparted to oral inflow 108. This change in air pulse frequency disrupts or cancels the vibration of soft tissue 102 at its resonant frequency. This disruption of vibration stops the person from snoring.

In an example, an airflow-adjusting mechanism may change the oscillation frequency of an oscillating member that is in fluid communication with an airflow channel through the airflow-constraining member. In various examples, an airflow-adjusting mechanism may adjust airflow characteristics and reduce snoring by changing the oscillating speed, resistance, frequency, momentum, or direction of an oscillating member such as a reed, flap, valve or ball. In various examples, the airflow-adjusting mechanism can change the oscillating speed, resistance, frequency, momentum, or direction of an oscillating member such as a reed, flap, valve or ball. In various examples, the airflow-adjusting mechanism may transduce the kinetic energy of airflow from human respiration into the oscillation of an oscillating member such as a reed, flap, valve or ball.

In various examples, this invention can be embodied in a device for reducing snoring comprising: (1) an airflow-constraining member that directs, reduces, or blocks respiratory airflow during sleep wherein this airflow-constraining member is selected from the group consisting of: a member inserted into a person's mouth; one or more members inserted into a person's nose; one or more members inserted into a person's mouth and nose; a member covering a person's mouth; a member covering a person's nose; and a member covering both a person's mouth and nose; (2) an airflow channel that allows airflow through or past the airflow-constraining member; and (3) an airflow-adjusting mechanism that enables adjustment of one or more airflow characteristics of airflow through the airflow channel in order to reduce or prevent vibration of soft tissue along the person's airway that causes snoring: (a) wherein these airflow characteristics are selected from the group consisting of: airflow speed, airflow rate, airflow pressure, airflow volume, airflow duration, airflow timing, airflow direction, the creation of one or more pulses of air, the number of air pulses, pattern of air pulses, the frequency of air pulses, the magnitude of air pulses, and the duration of air pulses; (b) wherein activation of adjustment of these airflow characteristics is selected from the group consisting of: manual adjustment of airflow characteristics by a person; and automatic adjustment of airflow characteristics while a person sleeps in response to the detection or prediction of snoring; (c) wherein the airflow-adjusting mechanism comprises one or more mechanisms selected from the group consisting of: changing the rotational speed, resistance, momentum, or direction of a rotating member such as a turbine, fan blade, impeller, paddlewheel, flywheel, helix, screw, or gear; changing the oscillating speed, resistance, frequency, momentum, or direction of an oscillating member such as a reed, flap, valve, ball, or speaker diaphragm; changing the shape, width, length, size, or closure of one or more airflow channels that allow airflow through the airflow-constraining member; changing the tension of an extendable or compressible member such as a spring or elastic band; changing the inflation of an expandable member such as a balloon; and changing the pressure within an air chamber; (d) wherein this airflow-adjusting mechanism enables adjustment of airflow characteristics in an iterative manner in order to select airflow characteristics that are successful in reducing or preventing snoring; and (e) wherein this airflow-adjusting mechanism transduces or harvests some of the kinetic energy of airflow from human respiration in order to adjust airflow characteristics in order to reduce the vibration of soft tissue.

In various examples, this invention can be embodied in a method for reducing snoring comprising: (1) constraining respiratory airflow using a airflow-constraining member that directs, reduces, or blocks respiratory airflow during sleep wherein this airflow-constraining member is selected from the group consisting of: a member inserted into a person's mouth; one or more members inserted into a person's nose; one or more members inserted into a person's mouth and nose; a member covering a person's mouth; a member covering a person's nose; and a member covering both a person's mouth and nose; (2) allowing airflow through or past the airflow-constraining member using an airflow channel; and (3) adjusting one or more characteristics of airflow through the airflow channel using an airflow-adjusting mechanism in order to reduce or prevent vibration of soft tissue along the person's airway that causes snoring: (a) wherein these airflow characteristics are selected from the group consisting of: airflow speed, airflow rate, airflow pressure, airflow volume, airflow duration, airflow timing, airflow direction, the creation of one or more pulses of air, the number of air pulses, pattern of air pulses, the frequency of air pulses, the magnitude of air pulses, and the duration of air pulses; (b) wherein activation of adjustment of these airflow characteristics is selected from the group consisting of: manual adjustment of airflow characteristics by a person; and automatic adjustment of airflow characteristics while a person sleeps in response to the detection or prediction of snoring; (c) wherein the airflow-adjusting mechanism comprises one or more mechanisms selected from the group consisting of: changing the rotational speed, resistance, momentum, or direction of a rotating member such as a turbine, fan blade, impeller, paddlewheel, flywheel, helix, screw, or gear; changing the oscillating speed, resistance, frequency, momentum, or direction of an oscillating member such as a reed, flap, valve, ball, or speaker diaphragm; changing the shape, width, length, size, or closure of one or more airflow channels that allow airflow through the airflow-constraining member; changing the tension of an extendable or compressible member such as a spring or elastic band; changing the inflation of an expandable member such as a balloon; and changing the pressure within an air chamber; (d) wherein this airflow-adjusting mechanism enables adjustment of airflow characteristics in an iterative manner in order to select airflow characteristics that are successful in reducing or preventing snoring; and (e) wherein this airflow-adjusting mechanism transduces or harvests some of the kinetic energy of airflow from human respiration in order to adjust airflow characteristics in order to reduce the vibration of soft tissue.

In an example, this invention includes an airflow-adjusting mechanism that is energy self-sufficient, wherein being energy self-sufficient is defined as being able to operate for at least a month without any source of external power and without having to recharge or replace any internal power source such as a battery, with the possible exception of any energy transduced or harvested from the kinetic energy of human respiration. In an example, this invention includes an airflow-adjusting mechanism that transduces or harvests the kinetic energy of airflow from human respiration into electrical energy that is stored and then used to adjust airflow characteristics in order to reduce vibration of soft tissue. In an example, this invention includes an airflow-adjusting mechanism wherein this airflow-adjusting mechanism is entirely located within twelve inches of the person's body.

In an example, this invention includes an airflow-adjusting mechanism wherein this airflow-adjusting mechanism creates a series of multiple air pulses that prevents, reduces, disrupts, or cancels the vibration of soft tissue at its resonant frequency.

In an example, this invention includes an airflow-adjusting mechanism wherein this airflow-adjusting mechanism changes the rotational speed, resistance, momentum, or direction of a turbine, fan blade, impeller, paddlewheel, flywheel, helix, screw, or gear. In an example, this invention includes an airflow-adjusting mechanism wherein this airflow-adjusting mechanism transduces some kinetic energy of airflow from human respiration into the rotation of a turbine, fan blade, impeller, paddlewheel, flywheel, helix, screw, or gear.

In an example, this invention includes an airflow-adjusting mechanism wherein this airflow-adjusting mechanism changes the oscillating speed, resistance, frequency, momentum, or direction of a reed, flap, valve, ball, or speaker diaphragm. In an example, this invention includes an airflow-adjusting mechanism wherein this airflow-adjusting mechanism transduces some kinetic energy of airflow from human respiration into the oscillation of a reed, flap, valve, ball, or speaker diaphragm.

In an example, this invention includes an airflow-adjusting mechanism wherein this airflow-adjusting mechanism changes the shape, width, length, size, or closure of one or more airflow channels that allow airflow through the airflow-constraining member. In an example, this invention includes an airflow-adjusting mechanism wherein this airflow-adjusting mechanism transduces some kinetic energy of airflow from human respiration into changes in the shape, width, length, size, or closure of one or more airflow channels that allow airflow through the airflow-constraining member.

In an example, this invention includes an airflow-adjusting mechanism wherein this airflow-adjusting mechanism changes the tension of a spring or elastic band. In an example, this invention includes an airflow-adjusting mechanism wherein this airflow-adjusting mechanism transduces some kinetic energy of airflow from human respiration into changes in the tension of a spring or elastic band.

In an example, this invention includes an airflow-adjusting mechanism wherein this airflow-adjusting mechanism changes the inflation of a balloon or changes the pressure within an air chamber. In an example, this invention includes an airflow-adjusting mechanism wherein this airflow-adjusting mechanism transduces some kinetic energy of airflow from human respiration into changes in the inflation of a balloon or changes in the pressure within an air chamber.

In an example, this invention includes an airflow-adjusting mechanism wherein this airflow-adjusting mechanism automatically adjusts airflow characteristics in an iterative manner in order to select airflow characteristics that are successful for reducing or preventing snoring. In an example, this invention includes an airflow-adjusting mechanism wherein this airflow-adjusting mechanism adjusts the relative differences between airflow characteristics of airflows through the mouth vs. nose in order to alter the combined aerodynamics of these flows on soft tissue along the airway.

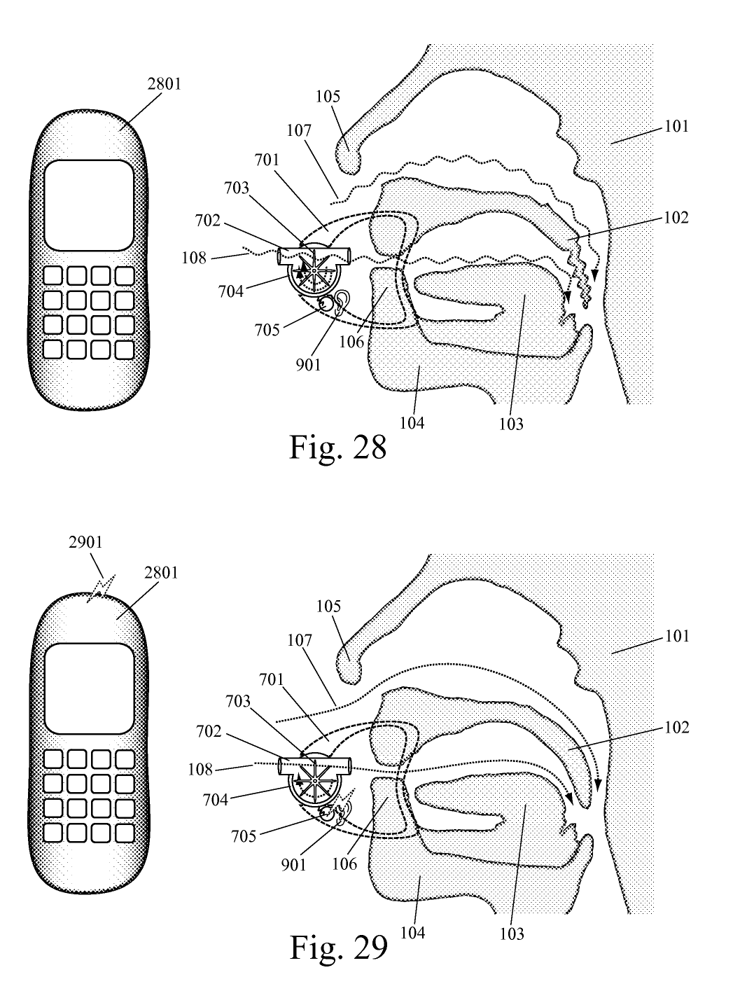

Virtually all of the examples of this invention that were shown in prior figures may be used with some type of wireless, or other remote, monitoring or control unit or function. However, to make this explicit, FIGS. 28-29 show one way in which the example of this invention that was shown previously in FIGS. 9-10 may be monitored and controlled by a wireless remote unit. FIG. 28 shows the same example that was shown in FIG. 9, but with the addition of wireless remote unit 2801.

FIG. 28 shows this device before airflow has been wirelessly adjusted, wherein the person is snoring. FIG. 29 shows a “lightning bolt” symbol that represents a wireless signal being transmitting from wireless remote unit 2801 to user interface 705 which triggers the adjustment of airflow characteristics and thereby stops snoring. Remote monitoring and/or control may be done in a manual manner, in an automatic manner, or in a manner which combines manual and automatic operations.

In various examples, this invention can include an airflow-adjusting mechanism that is in two-way communication with a wireless remote control or monitoring unit. In various examples, the airflow-adjusting mechanism may be in communication with a remote member selected from the group comprising: a wireless remote control or monitoring unit; a physically-connected remote control or monitoring unit; an application on a smart phone or other multi-purpose mobile device; and a remote internet website. In an example, this two-way communication may be via a local area network. In an example, this two-way communication may be via the worldwide internet. In an example, a remote monitoring and control unit may be used to monitor snoring and to adjust airflow in real time in order to reduce that snoring. In various examples, an airflow-adjusting mechanism may be in wireless communication with a remote control unit, a remote computer, or an internet-accessing device for remote monitoring and airflow adjustment to reduce snoring.

In an example, this invention includes an airflow-adjusting mechanism wherein this airflow-adjusting mechanism is in communication with a remote member selected from the group comprising: a wireless remote control or monitoring unit; a physically-connected remote control or monitoring unit; a smart phone or other multipurpose communication device; and an internet site. In an example, this invention includes an airflow-adjusting mechanism wherein this airflow-adjusting mechanism is in communication with a remote member that is used to monitor and adjust airflow characteristics in real time in order to reduce or prevent snoring.

In an example, this invention may include a wireless remote control that enables the snoring person's bed partner to adjust airflow characteristics in order to reduce the vibration of soft tissue. In an example, this invention may include a wireless remote control that enables someone other than the snoring person to adjust airflow characteristics in order to reduce the vibration of soft tissue. In an example, this invention may be embodied in a device and method that provides a remote control unit that allows a person to wirelessly adjust airflow characteristics of a device to reduce snoring by their bed partner who wears the anti-snoring device. Adjustment of airflow characteristics is limited to safe parameter intervals to ensure adequate fresh air. In an example, this device and method to reduce snoring can be self-contained (apart from the remote control unit), energy self-sufficient, and flexibly-adjustable over multiple respiratory cycles. This device and method offer couples a far better option than having the non-snoring bed partner repeatedly nudge or jab the snoring bed partner throughout the night. In an example, this invention may be called Adjustable Snore-Attenuating Pressure (ASAP) and appeal to people who want to stop snoring ASAP.

I claim:

1. A device for reducing snoring comprising:
an airflow-constraining member that directs, reduces, or blocks respiratory airflow during sleep wherein this airflow-constraining member is selected from the group consisting of: a member inserted into a person's mouth; one or more members inserted into a person's nose; one or more members inserted into a person's mouth and nose; a member covering a person's mouth; a member covering a person's nose; and a member covering both a person's mouth and nose;
an airflow channel that allows airflow through or past the airflow-constraining member; and
an airflow-adjusting mechanism that enables adjustment of one or more airflow characteristics of airflow through the airflow channel in order to reduce or prevent vibration of soft tissue along the person's airway that causes snoring: (a) wherein these airflow characteristics are selected from the group consisting of: airflow speed, airflow rate, airflow pressure, airflow volume, airflow duration, airflow timing, airflow direction, the creation of one or more pulses of air, the number of air pulses, pattern of air pulses, the frequency of air pulses, the magnitude of air pulses, and the duration of air pulses; (b) wherein activation of adjustment of these airflow characteristics is selected from the group consisting of: manual adjustment of airflow characteristics by a person; and
automatic adjustment of airflow characteristics while a person sleeps in response to the detection or prediction of snoring; (c) wherein the airflow-adjusting mechanism comprises one or more mechanisms selected from the group consisting of: changing the rotational speed, resistance, momentum, or direction of a rotating member such as a turbine, fan blade, impeller, paddlewheel, flywheel, helix, screw, or gear; changing the oscillating speed, resistance, frequency, momentum, or direction of an oscillating member such as a reed, flap, valve, ball, or speaker diaphragm; changing the shape, width, length, size, or closure of one or more airflow channels that allow airflow through the airflow-constraining member; changing the tension of an extendable or compressible member such as a spring or elastic band; changing the inflation of an expandable member such as a balloon; and changing the pressure within an air chamber; (d) wherein this airflow-adjusting mechanism enables adjustment of airflow characteristics in an iterative manner in order to select airflow characteristics that are successful in reducing or preventing snoring; and (e) wherein this airflow-adjusting mechanism transduces or harvests some of the kinetic energy of airflow from human respiration in order to adjust airflow characteristics in order to reduce the vibration of soft tissue.

2. The airflow-adjusting mechanism in claim 1 wherein this airflow-adjusting mechanism is energy self-sufficient, wherein being energy self-sufficient is defined as being able to operate for at least a month without any source of external power and without having to recharge or replace any internal power source such as a battery, with the possible exception of any energy transduced or harvested from the kinetic energy of human respiration.

3. The airflow-adjusting mechanism in claim 1 wherein this airflow-adjusting mechanism transduces or harvests the kinetic energy of airflow from human respiration into electrical energy that is stored and then used to adjust airflow characteristics in order to reduce vibration of soft tissue.

4. The airflow-adjusting mechanism in claim 1 wherein this airflow-adjusting mechanism creates a series of multiple air pulses that prevents, reduces, disrupts, or cancels the vibration of soft tissue at its resonant frequency.

5. The airflow-adjusting mechanism in claim 1 wherein this airflow-adjusting mechanism is entirely located within twelve inches of the person's body.

6. The airflow-adjusting mechanism in claim 1 wherein this airflow-adjusting mechanism changes the rotational speed, resistance, momentum, or direction of a turbine, fan blade, impeller, paddlewheel, flywheel, helix, screw, or gear.

7. The airflow-adjusting mechanism in claim 1 wherein this airflow-adjusting mechanism transduces some kinetic energy of airflow from human respiration into the rotation of a turbine, fan blade, impeller, paddlewheel, flywheel, helix, screw, or gear.

8. The airflow-adjusting mechanism in claim 1 wherein this airflow-adjusting mechanism changes the oscillating speed, resistance, frequency, momentum, or direction of a reed, flap, valve, ball, or speaker diaphragm.

9. The airflow-adjusting mechanism in claim 1 wherein this airflow-adjusting mechanism transduces some kinetic energy of airflow from human respiration into the oscillation of a reed, flap, valve, ball, or speaker diaphragm.

10. The airflow-adjusting mechanism in claim 1 wherein this airflow-adjusting mechanism changes the shape, width, length, size, or closure of one or more airflow channels that allow airflow through the airflow-constraining member.

11. The airflow-adjusting mechanism in claim 1 wherein this airflow-adjusting mechanism transduces some kinetic energy of airflow from human respiration into changes in the shape, width, length, size, or closure of one or more airflow channels that allow airflow through the airflow-constraining member.

12. The airflow-adjusting mechanism in claim 1 wherein this airflow-adjusting mechanism changes the tension of a spring or elastic band.

13. The airflow-adjusting mechanism in claim 1 wherein this airflow-adjusting mechanism transduces some kinetic energy of airflow from human respiration into changes in the tension of a spring or elastic band.

14. The airflow-adjusting mechanism in claim 1 wherein this airflow-adjusting mechanism changes the inflation of a balloon or changes the pressure within an air chamber.

15. The airflow-adjusting mechanism in claim 1 wherein this airflow-adjusting mechanism transduces some kinetic energy of airflow from human respiration into changes in the inflation of a balloon or changes in the pressure within an air chamber.

16. The airflow-adjusting mechanism in claim 1 wherein this airflow-adjusting mechanism is in communication with a remote member selected from the group comprising: a wireless remote control or monitoring unit; a physically-connected remote control or monitoring unit; a smart phone or other multipurpose communication device; and an internet site.

17. The airflow-adjusting mechanism in claim 1 wherein this mechanism automatically adjusts airflow characteristics in an iterative manner in order to select airflow characteristics that are successful for reducing or preventing snoring.

18. The airflow-adjusting mechanism in claim 1 wherein this airflow-adjusting mechanism enables adjustment of airflow characteristics in an iterative manner over the span of multiple respiratory cycles in order to select airflow characteristics that are successful in reducing or preventing snoring.

19. A method for reducing snoring comprising:
constraining respiratory airflow using a airflow-constraining member that directs, reduces, or blocks respiratory airflow during sleep wherein this airflow-constraining member is selected from the group consisting of: a member inserted into a person's mouth; one or more members inserted into a person's nose; one or more members inserted into a person's mouth and nose; a member covering a person's mouth; a member covering a person's nose; and a member covering both a person's mouth and nose;
allowing airflow through or past the airflow-constraining member using an airflow channel; and
adjusting one or more characteristics of airflow through the airflow channel using an airflow-adjusting mechanism in order to reduce or prevent vibration of soft tissue along the person's airway that causes snoring: (a) wherein these airflow characteristics are selected from the group consisting of: airflow speed, airflow rate, airflow pressure, airflow volume, airflow duration, airflow timing, airflow direction, the creation of one or more pulses of air, the number of air pulses, pattern of air pulses, the frequency of air pulses, the magnitude of air pulses, and the duration of air pulses; (b) wherein activation of adjustment of these airflow characteristics is selected from the group consisting of: manual adjustment of airflow characteristics by a person; and automatic adjustment of airflow characteristics while a person sleeps in response to the detection or prediction of snoring; (c) wherein the airflow-adjusting mechanism comprises one or more mechanisms selected from the group consisting of: changing the rotational speed, resistance, momentum, or direction of a rotating member such as a turbine, fan blade, impeller, paddlewheel, flywheel, helix, screw, or gear; changing the oscillating speed, resistance, frequency, momentum, or direction of an oscillating member such as a reed, flap, valve, ball, or speaker diaphragm; changing the shape, width, length, size, or closure of one or more airflow channels that allow airflow through the airflow-constraining member; changing the tension of an extendable or compressible member such as a spring or elastic band; changing the inflation of an expandable member such as a balloon; and changing the pressure within an air chamber; (d) wherein this airflow-adjusting mechanism enables adjustment of airflow characteristics in an iterative manner in order to select airflow characteristics that are successful in reducing or preventing snoring; and (e) wherein this airflow-adjusting mechanism transduces or harvests some of the kinetic energy of airflow from human respiration in order to adjust airflow characteristics in order to reduce the vibration of soft tissue.

20. A device for reducing snoring comprising:
an airflow-constraining member that directs, reduces, or blocks respiratory airflow during sleep wherein this airflow-constraining member is selected from the group consisting of: a member inserted into a person's mouth; one or more members inserted into a person's nose; one or more members inserted into a person's mouth and nose; a member covering a person's mouth; a member covering a person's nose; and a member covering both a person's mouth and nose;
an airflow channel that allows airflow through or past the airflow-constraining member;
an airflow-adjusting mechanism that enables adjustment of one or more airflow characteristics of airflow through the airflow channel in order to reduce or prevent vibration of soft tissue along the person's airway that causes snoring: (a) wherein these airflow characteristics are selected from the group consisting of: airflow speed, airflow rate, airflow pressure, airflow volume, airflow duration, airflow timing, airflow direction, the creation of one or more pulses of air, the number of air pulses, pattern of air pulses, the frequency of air pulses, the magnitude of air pulses, and the duration of air pulses; (b) wherein activation of adjustment of these airflow characteristics is selected from the group consisting of: manual adjustment of airflow characteristics by a person; and automatic adjustment of airflow characteristics while a person sleeps in response to the detection or prediction of snoring; (c) wherein the airflow-adjusting mechanism comprises one or more mechanisms selected from the group consisting of: changing the rotational speed, resistance, momentum, or direction of a rotating member such as a turbine, fan blade, impeller, paddlewheel, flywheel, helix, screw, or gear; changing the oscillating speed, resistance, frequency, momentum, or direction of an oscillating member such as a reed, flap, valve, ball, or speaker diaphragm; changing the shape, width, length, size, or closure of one or more airflow channels that allow airflow through the airflow-constraining member; changing the tension of an extendable or compressible member such as a spring or elastic band; changing the inflation of an expandable member such as a balloon; and changing the pressure within an air chamber; and (d) wherein this airflow-adjusting mechanism enables adjustment of airflow characteristics in an iterative manner in order to select airflow characteristics that are successful in reducing or preventing snoring; and
a remote control that enables the snoring person's bed partner to adjust airflow characteristics in order to reduce the vibration of soft tissue.
